SEQUENCE LISTING (3 sequences)

SEQ ID NO: 1 - protein, 583 aa, Flavobacterium okeanokoites
MFLSMVSKIRTFGWVQNPGKFENLKRVVQVFDRNSKVHNEVKNIKIPTLVKESKIQKELVAIMNQHDLIYTYKELVGTGTSIRSEAPCDAIIQATIADQGNKKGYIDNWSSDGFLRWAHALGFIEYINKSDSFVITDVGLAYSKSADGSAIEKEILIEAISSYPPAIRILTLLEDGQHLTKFDLGKNLGFSGESGFTSLPEGILLDTLANAMPKDKGEIRNNWEGSSDKYARMIGGWLDKLGLVKQGKKEFIIPTLGKPDNKEFISHAFKITGEGLKVLRRAKGSTKFTRVPKRVYWEMLATNLTDKEYVRTRRALILEILIKAGSLKIEQIQDNLKKLGFDEVIETIENDIKGLINTGIFIEIKGRFYQLKDHILQFVIPNRGVTKQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINF

SEQ ID NO: 2 - DNA, 53 bp, Unknown
  Description of Unknown Genomic target sequence
cctccgagaacgtcatcaccgagttcatgcgcttcaaggtgcgcatggagggc

SEQ ID NO: 3 - DNA, 13 bp, Unknown
  Description of Unknown Genomic target sequence
tctgacagatgac

(19) United States
(12) Patent Application Publication
(10) Pub. No.: US 2019/0359977 A1
(43) Pub. Date: Nov. 28, 2019

(54) Methods and Platform of Designing Genetic Editing Tools

(71) Applicant: Altius Institute for Biomedical Sciences, Seattle, WA (US)

(72) Inventors: Daniel Chee, Seattle, WA (US); Alister Funnell, Seattle, WA (US); Shreeram Akilesh, Seattle, WA (US); John Stamatoyannopoulos, Seatt, WA (US)

(21) Appl. No.: 16/480,246

(22) PCT Filed: Jan. 25, 2018
(86) PCT No.: PCT/US18/15328

§ 371 Date: Jul. 23, 2019

Related U.S. Application Data
(60) Provisional application No. 62/450,503, filed on Jan. 25, 2017.

(51) Int. Cl. C12N 15/10; C40B 30/04; C40B 40/06; G16B 30/00; G01N 21/78; G01N 21/64; G16B 40/10; C12N 9/22

(52) U.S. Cl. CPC C12N 15/1089; C40B 30/04; C40B 40/06; C12N 9/22; G01N 21/78; G01N 21/64; G16B 40/10; G16B 30/00

(57) ABSTRACT

This application provides a system and related methods that determine residue sequences for engineered proteins that facilitate genome engineering, including transcription activator-like effector nucleases. The system may receive an input DNA sequence for a region of a given genome and desired cleavage positions within the region. The system may determine candidate residue sequences for proteins that bind to the region and cleave the region at the desired cleavage positions, such as transcription activator-like effector nucleases (TALENs). The determination may be based on how the proteins may interact with the region and perform other biological functions. A selection can be made from the candidate residue sequences to achieve high accuracy and efficiency in the genome engineering tasks. The system may thus allow development of proteins that incorporate the selected residue sequences to perform the genome engineering tasks.

### CROSS-REFERENCE

This application claims the benefit of U.S. Provisional Patent Application No. 62/450,503, filed Jan. 25, 2017, the entire disclosure of which is incorporated by reference herein.

### SEQUENCE LISTING

The instant application contains a Sequence Listing that has been submitted electronically in ASCII format and is hereby incorporated by reference in its entirety. Said ASCII copy, created on Jan. 19, 2018, is named 48539-702_601_SL.txt and is 5,743 bytes in size.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BACKGROUND OF THE DISCLOSURE

Transcription activator-like effector nucleases (TALENs) are restriction enzymes that can be engineered to cut specific sequences of DNA. The restriction enzymes can be introduced into cells, for use in gene editing or for genome editing in situ.

### SUMMARY OF THE DISCLOSURE

In some aspects, provided herein are methods and platforms for generating a nucleic acid construct comprising a plurality of polynucleotides of interest. In some instances, also provided herein is a method of generating a transcription activator-like (TAL) effector endonuclease monomer (e.g., by a high-throughput method). In additional aspects, provided herein are isolated and purified transcription activator-like (TAL) effector endonuclease plasmids.

In some aspects, provided herein are a system and related methods that determine residue sequences for engineered proteins that facilitate genome engineering, including transcription activator-like effector nucleases. The system may receive an input DNA sequence for a region of a given genome and desired cleavage positions within the region. The system may determine candidate residue sequences for proteins that bind to the region and cleave the region at the desired cleavage positions, such as transcription activator-like effector nucleases. The determination may be based on how the proteins may interact with the region and/or perform other biological functions. A selection can be made from the candidate residue sequences to achieve high accuracy and efficiency in the genome engineering tasks. The system thus may allow the development of proteins that incorporate the selected residue sequences to perform the genome engineering tasks.

By pre-scanning a given genome sequence, the system may be able to quickly identify potential binding sites in any region within the genome. By scoring protein sequences based on their known or expected biological activity, the system may be able to determine which proteins to develop to accomplish the intended genome engineering tasks effectively. Overall, the efficient and extensive nature of the sequence determination performed by the system for transcription activator-like effector nucleases in particular may significantly facilitate engineering of human genomes and understanding of human life.

Disclosed herein, in an aspect, is a computer-implemented method of determining protein sequences for genome engineering, comprising: receiving input information regarding an input DNA sequence for a DNA region in a given genome containing binding sites for proteins and a cleavage position for the proteins within the DNA region; identifying a plurality of fragments of the input DNA sequence respectively corresponding to a plurality of the binding sites to a first side of the cleavage position; determining a plurality of protein di-residue sequences for a plurality of the proteins to bind to the plurality of binding sites based on specificity information related to binding of protein di-residues to DNA bases; assigning a score to each of the plurality of protein di-residue sequences with a scoring function that generates a score based on at least one of the following conditions of the protein di-residue sequence: (a) TALE length or number of repeats; (b) spacer length; (c) last repeat variable dinucleotide (RVD); (d) GC content of RVDs; (e) first RVDs; (f) uniqueness of binding sites in the given genome; or (g) number of mononucleotide repeats; and generating output information regarding the plurality of protein di-residue sequences, including the assigned scores. In some embodiments, the scoring function generates the score based on at least two of the conditions (a) through (g). In some embodiments, the scoring function generates the score based on at least three of the conditions (a) through (g). In some embodiments, the scoring function generates the score based on at least four of the conditions (a) through (g). In some embodiments, the scoring function generates the score based on at least five of the conditions (a) through (g). In some embodiments, the scoring function generates the score based on at least six of the conditions (a) through (g). In some embodiments, the scoring function generates the score based on all the conditions (a) through (g). In some embodiments, the scoring function generates a higher score when the TALE length or number of repeats of the protein di-residue sequence is between about 14 and about 21. In some embodiments, the scoring function generates a higher score when the TALE length or number of repeats of the protein di-residue sequence is between about 15 and about 20. In some embodiments, the spacer length of the protein di-residue sequence comprises a distance from a corresponding binding site of the protein di-residue sequence to the cleavage position of the protein di-residue sequence. In some embodiments, the scoring function generates a higher score when the spacer length of the protein di-residue sequence is about 14 to about 16 base pairs. In some embodiments, the scoring function generates a higher score when the last repeat variable dinucleotide (RVD) of the protein di-residue sequence is “NG.” In some embodiments, the scoring function generates a higher score when the last repeat variable dinucleotide (RVD) of the protein di-residue sequence is not “NG” but corresponds to a “T” according to FIG. 4A. In some embodiments, the scoring function generates a higher score when the GC content of RVDs of the protein di-residue sequence comprises a number of RVDs of the protein di-residue sequence that correspond to a “G” or a “C.” In some embodiments, the scoring function generates a higher score when the GC content of RVDs of the protein di-residue sequence is about 1 to about 10 RVDs. In some embodiments, the scoring function generates a higher score when the GC content of RVDs of the protein di-residue sequence is about 3 to about 5 RVDs. In some embodiments, each of the first N RVDs of the protein di-residue sequence corresponds to a “G” or a “C.” In some embodiments, the scoring function generates a higher score when N is about 1 to about 10. In some embodiments, the scoring function generates a higher score when N is about 3 to about 5. In some embodiments, the scoring function generates a higher score when N is 5. In some embodiments, the uniqueness of binding sites in the given genome of the protein di-residue sequence comprises a number of corresponding binding sites in the given genome of the protein di-residue sequence. In some embodiments, the scoring function is inversely proportional to the uniqueness of binding sites in the given genome of the protein di-residue sequence. In some embodiments, the number of mononucleotide repeats comprises a length of any series of consecutive RVDs in the protein di-residue sequence that correspond to a “G” or a “C” or that correspond to a “T” or an “A.” In some embodiments, the scoring function is inversely proportional to the number of mononucleotide repeats of the protein di-residue sequence. In some embodiments, at least one of the conditions (a) through (g) is used as an initial filter applied to the plurality of protein di-residue sequences. In some embodiments, the input information includes a start position and an end position of the DNA region within the given genome. In some embodiments, each of the plurality of binding sites satisfies a length requirement and a location requirement. In some embodiments, each of the plurality of binding sites satisfies a leading nucleotide constraint and a trailing nucleotide constraint. In some embodiments, the identifying includes selecting the plurality of fragments using a pre-built nucleotide index for the given genome. In some embodiments, the determining includes setting a specificity threshold and disregarding any binding the specificity of which does not exceed the specificity threshold. In some embodiments, the scoring function generates a higher score when a smaller number of consecutive protein di-residues that bind to a “T” or an “A” nucleotide or to a “G” or “a “C” nucleotide, or a certain range for a length of the corresponding binding site. In some embodiments, the scoring function associates a weight with at least one of the conditions (a) through (g) in computing a score. In some embodiments, the output information includes one of the plurality of protein di-residue sequences, a number of binding sites for the protein di-residue sequence in the DNA region or the given genome, or a start position for each of the binding sites in the DNA region or the given genome. In some embodiments, the computer-implemented method further comprises: identifying a second plurality of binding sites to the other side of the cleavage position within the DNA region; determining a second plurality of protein di-residue sequences for a second plurality of the proteins to bind to the second plurality of binding sites based on the specificity information; and assigning a score to each of the second plurality of protein di-residue sequences with the scoring function. In some embodiments, the computer-implemented method further comprises: repeating the identifying, the determining, and the assigning for a complementary DNA sequence of the input DNA sequence, wherein the output information includes one of the second plurality of protein di-residue sequences, a number of binding sites for the protein di-residue sequence in the DNA region or the given genome, or a start position for each of the binding sites in the DNA region or the given genome. In some embodiments, the computer-implemented method further comprises: selecting a first protein di-residue sequence out of the plurality of protein di-residue sequences and a second protein di-residue sequence out of the second plurality of protein di-residue sequences based on the assigned scores, wherein the first protein di-residue sequence has binding site that is a certain distance away to a first side of the cleavage position and the second protein di-residue sequence has a binding site that is the certain distance away to the other side of the cleavage location; and generating information regarding the selections of the first protein di-residue sequence and the second protein di-residue sequence. In some embodiments, wherein each of the proteins is a transcription activator-like effector nuclease, and wherein each of the protein di-residue sequences specifies the di-residues for the 12^{th} and the 13^{th} position of the loops in the transcription activator-like effector nuclease. In some embodiments, the method further comprises receiving the input information from a client device over a network, and sending the output information to the client device over the network. In some embodiments, the client device is a desktop computer, a laptop computer, a tablet, a cellular phone, or a wearable device.

Disclosed herein, in another aspect, is a non-transitory computer-readable storage medium with instructions stored thereon that, when executed by a computing system, cause the computing system to perform a method of determining protein sequences for genome engineering, the method comprising: receiving input information regarding an input DNA sequence for a DNA region in a given genome containing binding sites for proteins and a cleavage position for the proteins within the DNA region; identifying a plurality of fragments of the input DNA sequence respectively corresponding to a plurality of the binding sites to a first side of the cleavage position; determining a plurality of protein di-residue sequences for a plurality of the proteins to bind to the plurality of binding sites based on specificity information related to binding of protein di-residues to DNA bases; assigning a score to each of the plurality of protein di-residue sequences with a scoring function that generates a score based on at least one of the following conditions of the protein di-residue sequence: (a) TALE length or number of repeats; (b) spacer length; (c) last repeat variable dinucleotide (RVD); (d) GC content of RVDs; (e) first RVDs; (f) uniqueness of binding sites in the given genome; or (g) number of mononucleotide repeats; and sending output information regarding the plurality of protein di-residue sequences, including the assigned scores. In some embodiments, the method further comprises: computing a number of binding sites within the given genome for each of the plurality of protein di-residue sequences, wherein the plurality of conditions includes fewer binding sites within the given genome. In some embodiments, the computing is performed based on the specificity information. In some embodiments, the plurality of conditions includes a binding site having more “G” or “C” nucleotides. In some embodiments, the conditions include a protein di-residue that binds with a higher specificity or a protein di-residue that binds with a higher efficiency in promoting protein activity.

Disclosed herein, in another aspect, is a system for making nucleases for genome engineering, comprising: an apparatus that develops proteins; a memory; and at least one processor in communication with the memory and the apparatus, the processor configured to perform: receiving input information regarding an input DNA sequence for a DNA region in a given genome containing binding sites for proteins and a cleavage position for the proteins within the DNA region; identifying a plurality of fragments of each of the input DNA sequence and a complementary DNA sequence of the input DNA sequence respectively corresponding to a plurality of the binding sites to each of the two sides of the cleavage position within the DNA region; determining a plurality of protein di-residue sequences for a plurality of the proteins to bind to the plurality of binding sites based on specificity information related to binding of protein di-residues to DNA bases; assigning a score to each of the plurality of protein di-residue sequences with a scoring function that generates a score based on at least one of the following conditions of the protein di-residue sequence: (a) TALE length or number of repeats; (b) spacer length; (c) last repeat variable dinucleotide (RVD); (d) GC content of RVDs; (e) first RVDs; (f) uniqueness of binding sites in the given genome; or (g) number of mononucleotide repeats; and selecting, based on the assigned scores, a first protein di-residue sequence out of the pluralities of protein di-residue sequences corresponding to a protein that bind to the input DNA sequence to a first side of the cleavage position and a second protein di-residue sequence out of the pluralities of protein di-residue sequences that bind to the complementary DNA sequence to the other side of the cleavage position; and causing to display information regarding the first protein di-residue sequence and the second di-residue sequence, wherein the apparatus develops proteins based on the first and the second di-residue sequences.

Disclosed herein, in another aspect, is a computer-implemented method of determining protein sequences for genome engineering, comprising: receiving input information regarding an input DNA sequence for a DNA region in a given genome containing binding sites for proteins and a cleavage position for the proteins within the DNA region; identifying a plurality of fragments of the input DNA sequence respectively corresponding to a plurality of the binding sites to a first side of the cleavage position; determining a plurality of protein di-residue sequences for a plurality of the proteins to bind to the plurality of binding sites based on specificity information related to binding of protein di-residues to DNA bases; assigning a score to each of the plurality of protein di-residue sequences based on (1) a binding strength of initial protein di-residues, (2) a percentage of protein di-residues that bind to “G” or “C” nucleotides, or (3) a presence of consecutive protein di-residues that bind to “G” or “C” nucleotides or that bind to “A” or “T” nucleotides, in the protein di-residue sequence; and generating output information regarding the plurality of protein di-residue sequences, including the assigned scores. In some embodiments, the assigning includes calculating a score based on each of (1), (2), and (3), and determining a weighted average. In some embodiments, a higher score is assigned when more of a predetermined number of the initial protein di-residues form a strong bond with a target nucleotide. In some embodiments, a higher score is assigned when a larger percentage of the protein di-residues bind to “G” or “C” nucleotides. In some embodiments, a higher score is assigned when no more than a first predetermined number of consecutive protein di-residues bind to “G” or “C” nucleotides and no more than a second predetermined number of consecutive protein di-residues bind to “A” or “T” nucleotides. In some embodiments, a higher score is assigned when a length of the corresponding binding site falls in a first predetermined range or a length of a region between the corresponding binding site and the cleavage position falls in a second predetermined range. In some embodiments, the method further comprises receiving the input information from a client device over a network, and sending the output information to the client device over the network.

Disclosed herein, in another aspect, is a high-throughput method of generating a nucleic acid construct containing a plurality of polynucleotides of interest, comprising: (a) assembling a first plurality of polynucleotides of interest in a first reaction mixture comprising a plurality of first destination vectors; (b) incorporating the first plurality of polynucleotides of interest into at least one first destination vector from the plurality of first destination vectors by a nucleic acid incorporation process to generate at least one first expression vector, wherein the at least one first expression vector comprises a first polynucleotide unit, and wherein the first polynucleotide unit comprises the first plurality of polynucleotides of interest; (c) incubating the first reaction mixture comprising the at least one first expression vector from step b) with a first restriction enzyme to remove a first destination vector that fails to incorporate the first plurality of polynucleotides of interest; (d) repeating steps a) to c) with a second plurality of polynucleotides of interest and a plurality of second destination vectors to generate at least one second expression vector, wherein the at least one second expression vector comprises a second polynucleotide unit, and wherein the second polynucleotide unit comprises the second plurality of polynucleotides of interest; (e) assembling the at least one first expression vector and the at least one second expression vector with a third destination vector in a second reaction mixture; and (f) incorporating the first polynucleotide unit and the second polynucleotide unit from the at least one first expression vector and the at least one second expression vector into the third destination vector by said nucleic acid incorporation process to generate the nucleic acid construct containing a plurality of polynucleotides of interest. In some embodiments, the first restriction enzyme comprises BsaI or BsaI-HF. In some embodiments, the method further comprises incubating the first reaction mixture of step c) with a deoxyribonuclease. In some embodiments, the incubating of step c) is for at least 30 minutes, at least 40 minutes, at least 50 minutes, at least 60 minutes, at least 70 minutes, at least 80 minutes, at least 90 minutes, at least 2 hours, at least 3 hours, at least 4 hours, at least 5 hours, at least 6 hours, at least 10 hours, at least 12 hours, or more. In some embodiments, the incubating of step c) is at a temperature of about 37° C. In some embodiments, the incubating of step c) further comprises a transformation step, a culturing step, and a plasmid harvesting step. In some embodiments, the plasmid obtained from the plasmid harvesting step is further quantified by a spectrophotometric method. In some embodiments, the method further comprises incubating the second reaction mixture after step f) with a second restriction enzyme to remove a third destination vector that fails to incorporate the first polynucleotide unit and the second polynucleotide unit. In some embodiments, the second restriction enzyme comprises BsaI or BsaI-HF. In some embodiments, the method further comprises incubating the second reaction mixture after step f) with a deoxyribonuclease. In some embodiments, the incubating of the second reaction mixture after step f) is for at least 30 minutes, at least 40 minutes, at least 50 minutes, at least 60 minutes, at least 70 minutes, at least 80 minutes, at least 90 minutes, at least 2 hours, at least 3 hours, at least 4 hours, at least 5 hours, at least 6 hours, at least 10 hours, at least 12 hours, or more. In some embodiments, the incubating of the second reaction mixture after step f) is at a temperature of about 37° C. In some embodiments, the incubating further comprises a transformation step, a culturing step, and a plasmid harvesting step. In some embodiments, the nucleic acid incorporation process comprises at least one round of a digestion step and a ligation step. In some embodiments, the nucleic acid incorporation process comprises about 2, 3, 4, 5, 6, 7, 8, 9, 10, 15, 20, or more rounds of a digestion step and a ligation step. In some embodiments, the digestion step is at about 37° C. In some embodiments, the ligation step is at about 16° C. In some embodiments, the time for the digestion step is at least 2, 3, 4, 5, 6, 7, 8, 9, 10, 15, 30, or more minutes per round. In some embodiments, the time for the ligation step is about 5, 6, 7, 8, 9, 10, 15, 30, 45, 60, or more minutes per round. In some embodiments, the nucleic acid incorporation process further comprises a background reduction step. In some embodiments, the background reduction step occurs after at least one round of a digestion step and a ligation step. In some embodiments, the background reduction step occurs at a temperature of about 45° C., 50° C., 55° C., 60° C., or higher. In some embodiments, the time for the background reduction step is about 5, 10, 15, 20, or more minutes. In some embodiments, the nucleic acid incorporation process further comprises a heat inactivation step. In some embodiments, the heat inactivation step occurs at a temperature of about 65° C., 70° C., 75° C., 80° C., 85° C., 90° C., or higher. In some embodiments, the time for the heat inactivation step is about 5, 10, 15, 20, or more minutes. In some embodiments, the first plurality of polynucleotides of interest comprises a plurality of TAL effector repeat modules or a plurality of zinc-binding repeat modules. In some embodiments, the first plurality of polynucleotides of interest comprises a plurality of TAL effector repeat modules. In some embodiments, the first plurality of polynucleotides of interest comprises a plurality of polynucleotides for generating a fusion polypeptide or a plurality of polynucleotides in which each polynucleotide encodes a portion of a protein of interest. In some embodiments, the second plurality of polynucleotides of interest comprises a plurality of TAL effector repeat modules or a plurality of zinc-binding repeat modules. In some embodiments, the second plurality of polynucleotides of interest comprises a plurality of TAL effector repeat modules. In some embodiments, the second plurality of polynucleotides of interest comprises a plurality of polynucleotides for generating a fusion polypeptide or a plurality of polynucleotides in which each polynucleotide encodes a portion of a protein of interest. In some embodiments, the incorporating in step b) of the method further comprises incubating the plurality of TAL effector repeat modules and the at least one first destination vector in the first reaction mixture for a first time period. In some embodiments, the incorporating in step b) of the method further comprises culturing the plurality of TAL effector repeat modules and the at least one first destination vector for a second time period to generate a first TAL effector repeat containing vector. In some embodiments, step d) of the method further comprises generating a second TAL effector repeat containing vector from a second plurality of TAL effector repeat modules and the at least one second destination vector. In some embodiments, the incorporating in step f) of the method further comprises incubating the first and the second TAL effector repeat containing vectors and the third destination vector in the second reaction mixture for a third time period. In some embodiments, the incorporating in step f) of the method further comprises culturing the first and the second TAL effector repeat containing vectors and the third destination vector for a fourth time period to generate a transcription activator-like (TAL) effector endonuclease monomer. In some embodiments, the transcription activator-like (TAL) effector endonuclease monomer further comprises a FokI endonuclease domain and optionally a linker region. In some embodiments, the transcription activator-like (TAL) effector endonuclease monomer further comprises a N-cap and a C-cap. In some embodiments, the transcription activator-like (TAL) effector endonuclease monomer further comprises a C-terminal half-repeat. In some embodiments, the C-terminal half-repeat comprises about 15, 20, 21, 22, 23, 24, 25, 26, 27, 28, 29, 30, 35, or 40 amino acid residues. In some embodiments, a sequence encoding the C-terminal half-repeat is present within the third destination vector. In some embodiments, the transcription activator-like (TAL) effector endonuclease monomer further comprises a T base recognizing repeat variable-diresidue (RVD) at the N-terminal portion of the TAL effector repeat modules, at the C-terminal portion of the TAL effector repeat modules, or at both termini. In some embodiments, the insertion of the TAL effector repeat modules removes a LacZ portion of the second vector. In some embodiments, the plurality of TAL effector repeat modules comprises at least 1, 2, 3, 4, 5, 6, 7, 8, 9, 10, 11, 12, 13, 14, 15, 16, 17, 18, 19, 20, 21, 22, 23, 24, 25, 30, or more TAL effector repeat modules. In some embodiments, each of the plurality of TAL effector repeat modules comprises a repeat variable-diresidue (RVD). In some embodiments, the repeat variable-diresidue (RVD) comprises HD, NG, NI, NK, or NH. In some embodiments, the first destination vector is pFUS vector. In some embodiments, the first destination vector is pUC18 or pUC19 vector. In some embodiments, the second destination vector is pFUS vector. In some embodiments, the second destination vector is pUC18 or pUC19 vector. In some embodiments, the third destination vector is pVax vector. In some embodiments, the volume of the first reaction mixture is about 2 μL. In some embodiments, the volume of the second reaction mixture is about 2 μL. In some embodiments, the assembling of step a) and step e) are by an acoustic process. In some embodiments, the acoustic process is generated by a Labcyte Echo 550 high-throughput acoustic liquid handler instrument.

Disclosed herein, in another aspect, is a transcription activator-like (TAL) effector endonuclease monomer generated by the steps of: (a) assembling a first plurality of TAL effector repeat sequences in a first reaction mixture comprising a plurality of first destination vectors; (b) incorporating the first plurality of TAL effector repeat sequences into at least one first destination vector from the plurality of first destination vectors by a nucleic acid incorporation process to generate at least one first expression vector, wherein the at least one first expression vector comprises a first TAL effector repeat unit and wherein the first TAL effector repeat unit comprises the first plurality of TAL effector repeat sequences; (c) incubating the first reaction mixture comprising the at least one first expression vector from step b) with a first restriction enzyme to remove a first destination vector that fails to incorporate the first plurality of TAL effector repeat sequences; (d) repeating steps a) to c) with a second plurality of TAL effector repeat sequences and a plurality of second destination vectors to generate at least one second expression vector, wherein the at least one second expression vector comprises a second TAL effector repeat unit and wherein the second TAL effector repeat unit comprises the second plurality of TAL effector repeat sequences; (e) assembling the at least one first expression vector and the at least one second expression vector with a third destination vector in a second reaction mixture; and (f) incorporating the first TAL effector repeat unit and the second TAL effector repeat unit from the at least one first expression vector and the at least one second expression vector into the third destination vector by said nucleic acid incorporation process to generate the nucleic acid construct containing the transcription activator-like (TAL) effector endonuclease monomer.

Disclosed herein, in another aspect, is a high-throughput method of generating a nucleic acid construct containing a plurality of polynucleotides of interest, comprising: (a) assembling a first plurality of polynucleotides of interest and a plurality of first destination vectors in a first reaction mixture by an acoustic process; (b) incorporating the first plurality of polynucleotides of interest into at least one first destination vector from the plurality of first destination vectors by a nucleic acid incorporation process to generate at least one first expression vector, wherein the at least one first expression vector comprises a first polynucleotide unit and wherein the first polynucleotide unit comprises the first plurality of polynucleotides of interest; (c) repeating steps a) and b) with a second plurality of polynucleotides of interest and a plurality of second destination vectors to generate at least one second expression vector, wherein the at least one second expression vector comprises a second polynucleotide unit and wherein the second polynucleotide unit comprises the second plurality of polynucleotides of interest; (d) assembling the at least one first expression vector and the at least one second expression vector with a third destination vector in a second reaction mixture by said acoustic process; and (e) incorporating the first polynucleotide unit and the second polynucleotide unit from the at least one first expression vector and the at least one second expression vector into the third destination vector by said nucleic acid incorporation process to generate the nucleic acid construct containing a plurality of polynucleotides of interest. In some embodiments, the method further comprises a treating step after step b) but prior to step d), wherein the treating step comprises incubating the first reaction mixture from step b) with a first restriction enzyme to remove a first destination vector that fails to incorporate the first plurality of polynucleotides of interest. In some embodiments, the first restriction enzyme comprises BsaI or BsaI-HF. In some embodiments, the treating step further comprises incubating the first reaction mixture with a deoxyribonuclease. In some embodiments, the incubating is for at least 30 minutes, at least 40 minutes, at least 50 minutes, at least 60 minutes, at least 70 minutes, at least 80 minutes, at least 90 minutes, at least 2 hours, at least 3 hours, at least 4 hours, at least 5 hours, at least 6 hours, at least 10 hours, at least 12 hours, or more. In some embodiments, the incubating is at a temperature of about 37° C. In some embodiments, the treating step further comprises a transformation step, a culturing step, and a plasmid harvesting step. In some embodiments, the plasmid obtained from the plasmid harvesting step is further quantified by a spectrophotometric method. In some embodiments, the method further comprises a treating step after step e), wherein the treating step comprises incubating the second reaction mixture from step e) with a second restriction enzyme to remove a third destination vector that fails to incorporate the first polynucleotide unit and the second polynucleotide unit. In some embodiments, the second restriction enzyme comprises BsaI or BsaI-HF. In some embodiments, the treating step further comprises incubating the second reaction mixture after step f) with a deoxyribonuclease. In some embodiments, the incubating is for at least 30 minutes, at least 40 minutes, at least 50 minutes, at least 60 minutes, at least 70 minutes, at least 80 minutes, at least 90 minutes, at least 2 hours, at least 3 hours, at least 4 hours, at least 5 hours, at least 6 hours, at least 10 hours, at least 12 hours, or more. In some embodiments, the incubating is at a temperature of about 37° C. In some embodiments, the treating step further comprises a transformation step, a culturing step, and a plasmid harvesting step. In some embodiments, the first plurality of polynucleotides of interest comprises a plurality of TAL effector repeat modules or a plurality of zinc-binding repeat modules. In some embodiments, the first plurality of polynucleotides of interest comprises a plurality of TAL effector repeat modules. In some embodiments, the first plurality of polynucleotides of interest comprises a plurality of polynucleotides for generating a fusion polypeptide or a plurality of polynucleotides in which each polynucleotide encodes a portion of a protein of interest. In some embodiments, the second plurality of polynucleotides of interest comprises a plurality of TAL effector repeat modules or a plurality of zinc-binding repeat modules. In some embodiments, the second plurality of polynucleotides of interest comprises a plurality of TAL effector repeat modules. In some embodiments, the second plurality of polynucleotides of interest comprises a plurality of polynucleotides for generating a fusion polypeptide or a plurality of polynucleotides in which each polynucleotide encodes a portion of a protein of interest. In some embodiments, the incorporating in step b) of the method further comprises incubating the plurality of TAL effector repeat modules and the at least one first destination vector in the first reaction mixture for a first time period. In some embodiments, the incorporating in step b) of the method further comprises culturing the plurality of TAL effector repeat modules and the at least one first destination vector for a second time period to generate a first TAL effector repeat containing vector. In some embodiments, step c) of the method further comprises generating a second TAL effector repeat containing vector from a second plurality of TAL effector repeat modules and the at least one second destination vector. In some embodiments, the incorporating in step e) of the method further comprises incubating the first and the second TAL effector repeat containing vectors and the third destination vector in the second reaction mixture for a third time period. In some embodiments, the incorporating in step e) of the method further comprises culturing the first and the second TAL effector repeat containing vectors and the third destination vector for a fourth time period to generate a transcription activator-like (TAL) effector endonuclease monomer. In some embodiments, the transcription activator-like (TAL) effector endonuclease monomer further comprises a FokI endonuclease domain and optionally a linker region. In some embodiments, the transcription activator-like (TAL) effector endonuclease monomer further comprises a N-cap and a C-cap. In some embodiments, the transcription activator-like (TAL) effector endonuclease monomer further comprises a C-terminal half-repeat. In some embodiments, the C-terminal half-repeat comprises about 15, 20, 21, 22, 23, 24, 25, 26, 27, 28, 29, 30, 35, or 40 amino acid residues. In some embodiments, a sequence encoding the C-terminal half-repeat is present within the third destination vector. In some embodiments, the transcription activator-like (TAL) effector endonuclease monomer further comprises a T base recognizing-repeat variable-diresidue (RVD) at the N-terminal portion of the TAL effector repeat modules, at the C-terminal portion of the TAL effector repeat modules, or at both termini. In some embodiments, the insertion of the TAL effector repeat modules removes a LacZ portion of the second vector. In some embodiments, the plurality of TAL effector repeat modules comprises at least 1, 2, 3, 4, 5, 6, 7, 8, 9, 10, 11, 12, 13, 14, 15, 16, 17, 18, 19, 20, 21, 22, 23, 24, 25, 30, or more TAL effector repeat modules. In some embodiments, each of the plurality of TAL effector repeat modules comprises a repeat variable-diresidue (RVD). In some embodiments, the repeat variable-diresidue (RVD) comprises HD, NG, NI, NK, or NH. In some embodiments, the nucleic acid incorporation process comprises at least one round of a digestion step and a ligation step. In some embodiments, the nucleic acid incorporation process comprises about 2, 3, 4, 5, 6, 7, 8, 9, 10, 15, 20, or more rounds of a digestion step and a ligation step. In some embodiments, the digestion step is at about 37° C. In some embodiments, the ligation step is at about 16° C. In some embodiments, the time for the digestion step is at least 2, 3, 4, 5, 6, 7, 8, 9, 10, 15, 30, or more minutes per round. In some embodiments, the time for the ligation step is about 5, 6, 7, 8, 9, 10, 15, 30, 45, 60, or more minutes per round. In some embodiments, the nucleic acid incorporation process further comprises a background reduction step. In some embodiments, the background reduction step occurs after at least one round of a digestion step and a ligation step. In some embodiments, the background reduction step occurs at a temperature of about 45° C., 50° C., 55° C., 60° C., or higher. In some embodiments, the time for the background reduction step is about 5, 10, 15, 20, or more minutes. In some embodiments, the nucleic acid incorporation process further comprises a heat inactivation step. In some embodiments, the heat inactivation step occurs at a temperature of about 65° C., 70° C., 75° C., 80° C., 85° C., 90° C., or higher. In some embodiments, the time for the heat inactivation step is about 5, 10, 15, 20, or more minutes. In some embodiments, the first destination vector is pFUS vector. In some embodiments, the first destination vector is pUC18 or pUC19 vector. In some embodiments, the second destination vector is pFUS vector. In some embodiments, the second destination vector is pUC18 or pUC19 vector. In some embodiments, the third destination vector is pVax vector. In some embodiments, the volume of the first reaction mixture is about 2 μL. In some embodiments, the volume of the second reaction mixture is about 2 μL. In some embodiments, the acoustic process is generated by Labcyte Echo 550 high-throughput acoustic liquid handler instrument.

Disclosed herein, in another aspect, is a transcription activator-like (TAL) effector endonuclease monomer generated by the steps of: (a) assembling a first plurality of TAL effector repeat sequences and a plurality of first destination vectors in a first reaction mixture by an acoustic process; (b) incorporating the first plurality of TAL effector repeat sequences into at least one first destination vector from the plurality of first destination vectors by a nucleic acid incorporation process to generate at least one first expression vector, wherein the at least one first expression vector comprises a first TAL effector repeat unit and wherein the first TAL effector repeat unit comprises the first plurality of TAL effector repeat sequences; (c) repeating steps a) and b) with a second plurality of TAL effector repeat sequences and a plurality of second destination vectors to generate at least one second expression vector, wherein the at least one second expression vector comprises a second TAL effector repeat unit and wherein the second TAL effector repeat unit comprises the second plurality of TAL effector repeat sequences; (d) assembling the at least one first expression vector and the at least one second expression vector with a third destination vector in a second reaction mixture by said acoustic process; and (e) incorporating the first TAL effector repeat unit and the second TAL effector repeat unit from the at least one first expression vector and the at least one second expression vector into the third destination vector by said nucleic acid incorporation process to generate the transcription activator-like (TAL) effector endonuclease monomer.

Disclosed herein, in another aspect, is a method for making transcription activator-like effector nucleases (TALENs) for genome engineering, comprising: determining, by a computer-implemented method, scores for a plurality of protein di-residue sequences corresponding to an input DNA sequence for a DNA region in a given genome containing binding sites for proteins and a cleavage position for the proteins within the DNA region; selecting, based on the scores, a first protein di-residue sequence out of the plurality of protein di-residue sequences corresponding to a protein that bind to the input DNA sequence to a first side of the cleavage position and a second protein di-residue sequence out of the plurality of protein di-residue sequences that bind to the complementary DNA sequence to the other side of the cleavage position; and producing the TALENs based on the first and the second di-residue sequences.

### BRIEF DESCRIPTION OF THE DRAWINGS

Various aspects of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 represents a conceptual illustration of a method described herein.

FIG. 2 illustrates how transcription activator-like effector nucleases (TALENs) facilitate site-specific DNA sequence cleavage. FIG. 2 discloses SEQ ID NO: 2.

FIG. 3 illustrates the structure of a TALEN. FIG. 3 discloses SEQ ID NO: 3.

FIG. 4A shows a list of known repeat variable di-residues (RVDs) that bind to each of the possible nucleotides.

FIG. 4B shows the list of known RVDs together with known binding specificity and known binding efficiency.

FIG. 5 illustrates example computer components that can be used for implementing the system disclosed in this application.

FIG. 6 illustrates an example process performed by the system of generating a pair of transcription activator-like effector (TALE) RVD sequences for TALEN cleavage.

FIG. 7A illustrates an example application of TALEN cleavage for a single-hit task.

FIG. 7B illustrates an example application of TALEN cleavage for a flank/excision task.

FIG. 7C illustrates an example application of TALEN cleavage for a strafe task.

FIG. 7D illustrates an example application of TALEN cleavage for an imaging task.

FIG. 8 shows a computer system that can be configured to implement any computing system disclosed in the present application.

FIG. 9 illustrates an exemplary Echo Assembly protocol.

FIG. 10 illustrates an UV spectrophotometry measurement of DNA elutes from Day 2 culture samples.

FIG. 11 shows an exemplary electrophoresis gel analysis of 110 TALEN products.

### DETAILED DESCRIPTION OF THE DISCLOSURE

Design and assembly of a vector encoding a protein of interest from multiple plasmid units can be a time-intensive and cost-intensive process involving iterative steps of cloning starting plasmid units into intermediate plasmids and subsequent assembly of intermediate plasmids into the final vector while ensuring that plasmids are assembled correctly at each step. Described herein is a low-cost, high-throughput method of generating a nucleic acid construct of interest (e.g., encoding a plurality of polynucleotides of interest) and a computer implemented method and system for designing such constructs. The high-throughput methodology can enable assembly of a reaction mixture with reduced time and reduced volume of reagents, for example, at a volume of less than 5 μL, less than 4 μL, less than 3 μL, less than 2 μL, or less than 1 μL. The high-throughput methodology can also enable assembling of plasmids encoding a protein of interest with reduced background and with increased efficiency and yield. The computer-implemented method and system can enable construct designs across a region of interest, and without, for example, limitation on the length of the region, and can, based on an optimized scoring system, enable locating and optimizing a nucleic acid construct.

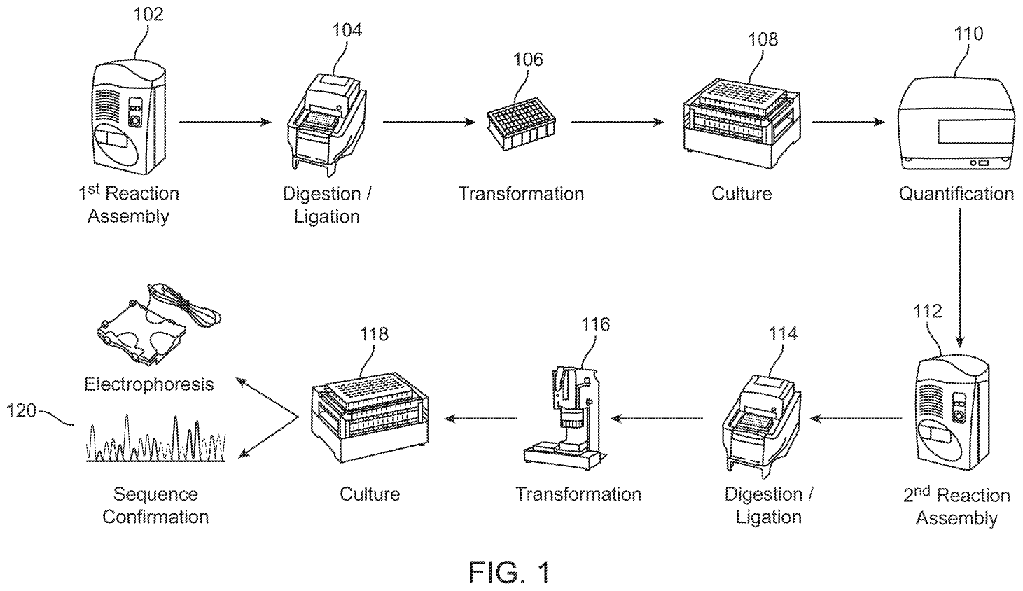

In some instances, a high-throughput method described herein is illustrated in FIG. 1. A reaction mixture can be assembled by an acoustic delivery system (e.g., utilizing a high-throughput acoustic liquid handler instrument, such as a Labcyte Echo 550) to assemble plasmids (e.g., encoding proteins of interest) en masse. The assembly can involve two steps: assembly of arrays of intermediary repeat units (e.g., about 1-10 or about 1-6 repeats per repeat unit) and joining of the intermediary arrays into a backbone vector to generate the final polypeptide of interest. The process can be completed in about 3, 4, or 5 days. In some instances, the process can be completed in about 3 days. In particular, a first reaction mixture can be assembled utilizing an acoustic delivery system on a microplate (102), with reduced reaction volumes (e.g., a volume of less than 5 μL, less than 4 μL, less than 3 μL, less than 2 μL or less than 1 μL). Upon assembly, the microplate can be further incubated in a thermocycler for about 10 or more cycles (104). Each cycle can comprise a digestion and a ligation step. A digestion step can take about 5 or more minutes at 37° C. and a ligation step can take about 10 or more minutes at 16° C. After each cycle, the reaction mixture is further heated to about 50° C. for about 5 or more minutes and then to about 80° C. for about 5 or more minutes to reduce background. After completion of the 10 or more cycles, a combination of a restriction enzyme and a deoxyribonuclease can be added into the reaction mixture to reduce background (e.g., empty vectors and/or unligated plasmids). The combination can be incubated with the first reaction mixture for at least 1 hour, 2 hours, 3 hours, 4 hours, or more at 37° C. The treated reaction mixture can be used to transform into host cells for amplification of a plasmid of interest (106). The transformed host cells can be grown for up to 20-24 hours (108) and subsequently processed and quantified (110). Those DNA concentrations above a certain threshold can be used in a second reaction assembly (112) to generate the final polypeptide of interest. Similar to the first reaction assembly, the second reaction assembly can be generated by an acoustic delivery system on a microplate (112) and can undergo a digestion/ligation cycle (114), transformation step (116) and a culturing step (118) to generate the final polypeptide of interest. Sequence confirmation and/or electrophoresis (120) can be used to determine a correctly assembled construct that encodes the polypeptide of interest.

### Polynucleotides of Interest

A high-throughput method provided herein can generate a nucleic acid construct that comprises a plurality of polynucleotides of interest. In some instances, a plurality of polynucleotides of interest comprises a plurality of TAL effector repeat modules or a plurality of zinc-binding repeat modules. In some cases, a plurality of polynucleotides of interest can comprise a plurality of polynucleotides for generating a fusion polypeptide or a plurality of polynucleotides in which each polynucleotide encodes a portion of a protein of interest. In some cases, a plurality of polynucleotides of interest comprises a plurality of TAL effector repeat modules. In other cases, a plurality of polynucleotides of interest comprises a plurality of zinc-binding repeat modules. In additional cases, a plurality of polynucleotides of interest comprises polynucleotides that encode one or more fusion polypeptides or a protein of interest.

### Transcription Activator-Like (TAL) Effector Nuclease Polypeptide

Transcription activator-like effector nuclease (TALEN) polypeptide is a restriction enzyme that can be engineered to target and edit specific nucleic acid sequences. TALEN can comprise a TAL effector DNA-binding domain fused to a nuclease domain. In some instances, TAL effector is a protein secreted from Xanthomonas bacteria upon plant infection. In some instances, TAL effector is a protein that is a mutated form of, or otherwise derived from, a protein secreted from Xanthomonas bacteria. TAL effector further comprises a DNA-binding module which includes a variable number of about 33-35 amino acid residue repeats. Each amino acid repeat recognizes one base pair through two adjacent amino acids (e.g., at amino acid positions 12 and 13 of the repeat). As such, the amino acid repeat can also be referred to as repeat-variable diresidue (RVD).

A TALEN described herein can comprise between about 1 to about 50 TAL effector repeat modules. A TALEN described herein can comprise between about 5 and about 45, between about 8 to about 45, between about 10 to about 40, between about 12 to about 35, between about 15 to about 30, between about 20 to about 30, between about 8 to about 40, between about 8 to about 35, between about 8 to about 30, between about 10 to about 35, between about 10 to about 30, between about 10 to about 25, between about 10 to about 20, or between about 15 to about 25 TAL effector repeat modules.

A TALEN described herein can comprise at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, or more TAL effector repeat modules. A TALEN described herein can comprise about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, or 50 TAL effector repeat modules. A TALEN described herein can comprise about 5 TAL effector repeat modules. A TALEN described herein can comprise about 10 TAL effector repeat modules. A TALEN described herein can comprise about 11 TAL effector repeat modules. A TALEN described herein can comprise about 12 TAL effector repeat modules. A TALEN described herein can comprise about 13 TAL effector repeat modules. A TALEN described herein can comprise about 14 TAL effector repeat modules. A TALEN described herein can comprise about 15 TAL effector repeat modules. A TALEN described herein can comprise about 16 TAL effector repeat modules. A TALEN described herein can comprise about 17 TAL effector repeat modules. A TALEN described herein can comprise about 18 TAL effector repeat modules. A TALEN described herein can comprise about 19 TAL effector repeat modules. A TALEN described herein can comprise about 20 TAL effector repeat modules. A TALEN described herein can comprise about 21 TAL effector repeat modules. A TALEN described herein can comprise about 22 TAL effector repeat modules. A TALEN described herein can comprise about 23 TAL effector repeat modules. A TALEN described herein can comprise about 24 TAL effector repeat modules. A TALEN described herein can comprise about 25 TAL effector repeat modules. A TALEN described herein can comprise about 26 TAL effector repeat modules. A TALEN described herein can comprise about 27 TAL effector repeat modules. A TALEN described herein can comprise about 28 TAL effector repeat modules. A TALEN described herein can comprise about 29 TAL effector repeat modules. A TALEN described herein can comprise about 30 TAL effector repeat modules. A TALEN described herein can comprise about 35 TAL effector repeat modules. A TALEN described herein can comprise about 40 TAL effector repeat modules. A TALEN described herein can comprise about 45 TAL effector repeat modules. A TALEN described herein can comprise about 50 TAL effector repeat modules.

A TAL effector repeat module can be a wild-type TAL effector DNA-binding module or a modified TAL effector DNA-binding repeat module enhanced for specific recognition of a nucleotide. A TALEN described herein can comprise one or more wild-type TAL effector DNA-binding module. A TALEN described herein can comprise one or more modified TAL effector DNA-binding repeat module enhanced for specific recognition of a nucleotide. A modified TAL effector DNA-binding repeat module can comprise 1, 2, 3, 4, 5, 6, 7, 8, 9, 10, 15, 20, 25 or more mutations that can enhance the repeat module for specific recognition of a nucleotide. In some cases, a modified TAL effector DNA-binding repeat module is modified at amino acid position 2, 3, 4, 11, 12, 13, 21, 23, 24, 25, 26, 27, 28, 30, 31, 32, 33, 34, or 35. In some cases, a modified TAL effector DNA-binding repeat module is modified at amino acid positions 12 or 13.

A TAL effector repeat module can be a repeat module-like domain or RVD-like domain. A RVD-like domain has a sequence different from naturally occurring polynucleotidic repeat module comprising RVD (RVD domain) but have a similar function and/or global structure. Non-limiting examples of RVD-like domains include protein domains selected from Puf RNA binding protein or Ankyrin super-family.

A TAL effector repeat module can be a RVD domain of Table 1. In some cases, a TALEN described herein can comprise one or more RVD domains selected from Table 1. In some cases, a TALEN described herein can comprise at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, or more RVD domains selected from Table 1.

TABLE 1

| RVD | Nucleotide |
|---|---|
| HD | C |
| NG | T |
| NI | A |
| NN | G > A |
| NS | G, A > C > T |
| NH | G |
| N* | T > C >> G, A |
| NP | T > A, C |
| HG | T |
| H* | T |
| IG | T |
| HA | C |
| ND | C |
| NK | G |
| HI | C |
| HN | G > A |
| NT | G > A |
| NA | G |
| SN | G or A |
| SH | G |
| YG | T |
| IS | — |

*Denotes a gap in the repeat sequence corresponding to a lack of an amino acid residue at the second position of the RVD.

In some cases, a RVD domain can recognize or interact with one nucleotide. Other times, a RVD domain can recognize or interact with more than one nucleotides. In some cases, the efficiency of a RVD domain at recognizing a nucleotide is ranked as “strong”, “intermediate” or “weak”. The ranking can be performed, for example, as described in Streubel et al., “TAL effector RVD specificities and efficiencies,” Nature Biotechnology 30(7): 593-595 (2012), which is incorporated herein by reference in its entirety. The ranking of RVD can be performed as illustrated in Table 2, for example, as described in Streubel et al., “TAL effector RVD specificities and efficiencies,” Nature Biotechnology 30(7): 593-595 (2012).

TABLE 2

| RVD | Nucleotide | Efficiency |
|---|---|---|
| HD | C | strong |
| NG | T | weak |
| NI | A | weak |
| NN | G > A | strong (G), |
| | | intermediate (A) |
| NS | G, A > C > T | intermediate |
| NH | G | intermediate |
| N* | T > C >> G, A | weak |
| NP | T > A, C | intermediate |
| NK | G | weak |
| HN | G > A | intermediate |
| NT | G > A | intermediate |
| SN | G or A | weak |
| SH | G | weak |
| IS | — | weak |

*Denotes a gap in the repeat sequence corresponding to a lack of an amino acid residue at the second position of the RVD.

A TAL effector DNA-binding domain can further comprise a C-terminal truncated TAL effector DNA-binding repeat module. A C-terminal truncated TAL effector DNA-binding repeat module can be between about 18 and about 40 residues in length. A C-terminal truncated TAL effector DNA-binding repeat module can be between about 20 to about 40, between about 22 to about 38, between about 24 to about 35, between about 28 to about 32, between about 25 to about 40, between about 25 to about 38, between about 25 to about 30, between about 28 to about 40, or between about 28 to about 35 residues in length. A C-terminal truncated TAL effector DNA-binding repeat module can be at least 18, 19, 20, 21, 22, 23, 24, 25, 26, 27, 28, 29, 30, 31, 32, 33, 34, 35, 36 37, 38, 39, or more residues in length. A C-terminal truncated TAL effector DNA-binding repeat module can be about 18, 19, 20, 21, 22, 23, 24, 25, 26, 27, 28, 29, 30, 31, 32, 33, 34, 35, 36 37, 38, 39 or 40 residues in length. A C-terminal truncated TAL effector DNA-binding repeat module can be about 18 residues in length. A C-terminal truncated TAL effector DNA-binding repeat module can be about 19 residues in length. A C-terminal truncated TAL effector DNA-binding repeat module can be about 20 residues in length. A C-terminal truncated TAL effector DNA-binding repeat module can be about 21 residues in length. A C-terminal truncated TAL effector DNA-binding repeat module can be about 22 residues in length. A C-terminal truncated TAL effector DNA-binding repeat module can be about 23 residues in length. A C-terminal truncated TAL effector DNA-binding repeat module can be about 24 residues in length. A C-terminal truncated TAL effector DNA-binding repeat module can be about 25 residues in length. A C-terminal truncated TAL effector DNA-binding repeat module can be about 26 residues in length. A C-terminal truncated TAL effector DNA-binding repeat module can be about 27 residues in length. A C-terminal truncated TAL effector DNA-binding repeat module can be about 28 residues in length. A C-terminal truncated TAL effector DNA-binding repeat module can be about 29 residues in length. A C-terminal truncated TAL effector DNA-binding repeat module can be about 30 residues in length. A C-terminal truncated TAL effector DNA-binding repeat module can be about 31 residues in length. A C-terminal truncated TAL effector DNA-binding repeat module can be about 32 residues in length. A C-terminal truncated TAL effector DNA-binding repeat module can be about 33 residues in length. A C-terminal truncated TAL effector DNA-binding repeat module can be about 34 residues in length. A C-terminal truncated TAL effector DNA-binding repeat module can be about 35 residues in length. A C-terminal truncated TAL effector DNA-binding repeat module can be about 36 residues in length. A C-terminal truncated TAL effector DNA-binding repeat module can be about 37 residues in length. A C-terminal truncated TAL effector DNA-binding repeat module can be about 38 residues in length. A C-terminal truncated TAL effector DNA-binding repeat module can be about 39 residues in length. A C-terminal truncated TAL effector DNA-binding repeat module can be about 40 residues in length. A C-terminal truncated TAL effector DNA-binding repeat module can be a RVD domain of Table 1.

A TAL effector DNA-binding domain can further comprise an N-terminal cap. An N-terminal cap can be a polypeptide portion flanking the DNA-binding repeat module. An N-terminal cap can be any length and can comprise from about 0 to about 136 amino acid residues in length. An N-terminal cap can be about 5, 10, 15, 20, 25, 30, 35, 40, 45, 50, 60, 70, 80, 90, 100, 110, 120, or 130 amino acid residues in length. In some instances, an N-terminal cap can modulate structural stability of the DNA-binding repeat modules. In some cases, an N-terminal cap can modulate nonspecific interactions. In some cases, an N-terminal cap can decrease nonspecific interaction. In some cases, an N-terminal cap can reduce off-target effect. As used here, off-target effect refers to the interaction of a TALEN with a sequence that is not the target sequence of interest. An N-terminal cap can further comprise a wild-type N-terminal cap sequence of a TALE protein or can comprise a modified N-terminal cap sequence.

A TAL effector DNA-binding domain can further comprise a C-terminal cap sequence. A C-terminal cap sequence can be a polypeptide portion flanking the C-terminal truncated TAL effector DNA-binding repeat module. A C-terminal cap can be any length and can comprise from about 0 to about 278 amino acid residues in length. A C-terminal cap can be about 5, 10, 15, 20, 25, 30, 35, 40, 45, 50, 60, 80, 100, 150, 200, or 250 amino acid residues in length. A C-terminal cap can further comprise a wild-type C-terminal cap sequence of a TALE protein, or can comprise a modified C-terminal cap sequence.

A nuclease domain fused to a TAL effector DNA-binding domain can be an endonuclease or an exonuclease. An endonuclease can include restriction endonucleases and homing endonucleases. An endonuclease can also include S1 Nuclease, mung bean nuclease, pancreatic DNase I, micrococcal nuclease, or yeast HO endonuclease. An exonuclease can include a 3′-5′ exonuclease or a 5′-3′ exonuclease. An exonuclease can also include a DNA exonuclease or an RNA exonuclease. Examples of exonuclease includes exonucleases I, II, III, IV, V, and VIII; DNA polymerase I, RNA exonuclease 2, and the like.

A nuclease domain fused to a TAL effector DNA-binding domain can be a restriction endonuclease (or restriction enzyme). In some instances, a restriction enzyme cleaves DNA at a site removed from the recognition site and has a separate binding and cleavage domains. In some instances, such restriction enzyme is a Type IIS restriction enzyme.

A nuclease domain fused to a TAL effector DNA-binding domain can be a Type IIS nuclease. A Type IIS nuclease can be FokI or Bfil. In some cases, a nuclease domain fused to a TAL effector DNA-binding domain is FokI. In other cases, a nuclease domain fused to a TAL effector DNA-binding domain is Bfil.

FokI can be a wild-type FokI or can comprise one or more mutations. In some cases, FokI can comprise about 1, 2, 3, 4, 5, 6, 7, 8, 9, 10, or more mutations. A mutation can enhance cleavage efficiency. A mutation can abolish cleavage activity. In some cases, a mutation can enhance homodimerization. For example, FokI can have a mutation at one or more amino acid residue positions 446, 447, 479, 483, 484, 486, 487, 490, 491, 496, 498, 499, 500, 531, 534, 537, and 538 to modulate homodimerization.

In some instances, a FokI cleavage domain is, for example, as described in Kim et al. “Hybrid restriction enzymes: Zinc finger fusions to Fok I cleavage domain,” PNAS 93: 1156-1160 (1996), which is incorporated herein by reference in its entirety. In some cases, a FokI cleavage domain described herein is a FokI of SEQ ID NO: 1 (Table 5). In other instances, a FokI cleavage domain described herein is a FokI, for example, as described in U.S. Pat. No. 8,586,526, which is incorporated herein by reference in its entirety.

A nuclease domain can be linked to a TAL effector DNA-binding domain either directly or through a linker. A linker can be between about 1 to about 50 amino acid residues in length. A linker can be from about 5 to about 45, from about 5 to about 40, from about 5 to about 35, from about 5 to about 30, from about 5 to about 25, from about 5 to about 20, from about 5 to about 15, from about 10 to about 40, from about 10 to about 35, from about 10 to about 30, from about 10 to about 25, from about 10 to about 20, from about 12 to about 40, from about 12 to about 35, from about 12 to about 30, from about 12 to about 25, from about 12 to about 20, from about 14 to about 40, from about 14 to about 35, from about 14 to about 30, from about 14 to about 25, from about 14 to about 20, from about 14 to about 16, from about 15 to about 40, from about 15 to about 35, from about 15 to about 30, from about 15 to about 25, from about 15 to about 20, from about 15 to about 18, from about 18 to about 40, from about 18 to about 35, from about 18 to about 30, from about 18 to about 25, from about 18 to about 24, from about 20 to about 40, from about 20 to about 35, from about 20 to about 30, or from about 25 to about 30 amino acid residues in length.

A linker for linking a nuclease domain to a TAL effector DNA-binding domain can be about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45 or 50 amino acid residues in length. A linker can be about 10 amino acid residues in length. A linker can be about 11 amino acid residues in length. A linker can be about 12 amino acid residues in length. A linker can be about 13 amino acid residues in length. A linker can be about 14 amino acid residues in length. A linker can be about 15 amino acid residues in length. A linker can be about 16 amino acid residues in length. A linker can be about 17 amino acid residues in length. A linker can be about 18 amino acid residues in length. A linker can be about 19 amino acid residues in length. A linker can be about 20 amino acid residues in length. A linker can be about 21 amino acid residues in length. A linker can be about 22 amino acid residues in length. A linker can be about 23 amino acid residues in length. A linker can be about 24 amino acid residues in length. A linker can be about 25 amino acid residues in length. A linker can be about 26 amino acid residues in length. A linker can be about 27 amino acid residues in length. A linker can be about 28 amino acid residues in length. A linker can be about 29 amino acid residues in length. A linker can be about 30 amino acid residues in length.

### Methods of Generating a TALEN

In some instances, a method of generating a transcription activator-like (TAL) effector endonuclease monomer is provided herein. In some cases, a TAL effector endonuclease monomer is generated with one or more methods described herein with reduced time and reduced volume of reagents, for example, at a volume of less than 5 μL, less than 4 μL, less than 3 μL, less than 2 μL or less than 1 μL. In some cases, a TAL effector endonuclease monomer is generated with one or more methods described herein with reduced background and with increased efficiency and yield. In additional cases, a TAL effector endonuclease monomer is generated with one or more methods described herein reduced intermediate steps.

In some instances, a method of generating a transcription activator-like (TAL) effector endonuclease monomer can comprise the steps of (a) assembling a first plurality of TAL effector repeat sequences in a first reaction mixture comprising a plurality of first destination vectors; (b) incorporating the first plurality of TAL effector repeat sequences into at least one first destination vector from the plurality of first destination vectors by a nucleic acid incorporation process to generate at least one first expression vector, wherein the at least one first expression vector comprises a first TAL effector repeat unit and wherein the first TAL effector repeat unit comprises the first plurality of TAL effector repeat sequences; (c) incubating the first reaction mixture comprising the at least one first expression vector from step b) with a first restriction enzyme to remove a first destination vector that fails to incorporate the first plurality of TAL effector repeat sequences; (d) repeating steps (a) to (c) with a second plurality of TAL effector repeat sequences and a plurality of second destination vectors to generate at least one second expression vector, wherein the at least one second expression vector comprises a second TAL effector repeat unit and wherein the second TAL effector repeat unit comprises the second plurality of TAL effector repeat sequences; (e) assembling the at least one first expression vector and the at least one second expression vector with a third destination vector in a second reaction mixture; and (f) incorporating the first TAL effector repeat unit and the second TAL effector repeat unit from the at least one first expression vector and the at least one second expression vector into the third destination vector by said nucleic acid incorporation process to generate the nucleic acid construct containing the transcription activator-like (TAL) effector endonuclease monomer.

In some cases, a method of generating a transcription activator-like (TAL) effector endonuclease monomer can comprise the step of a) assembling a first plurality of TAL effector repeat sequences and a plurality of first destination vectors in a first reaction mixture by an acoustic process; b) incorporating the first plurality of TAL effector repeat sequences into at least one first destination vector from the plurality of first destination vectors by a nucleic acid incorporation process to generate at least one first expression vector, wherein the at least one first expression vector comprises a first TAL effector repeat unit and wherein the first TAL effector repeat unit comprises the first plurality of TAL effector repeat sequences; c) repeating steps a) and b) with a second plurality of TAL effector repeat sequences and a plurality of second destination vectors to generate at least one second expression vector, wherein the at least one second expression vector comprises a second TAL effector repeat unit and wherein the second TAL effector repeat unit comprises the second plurality of TAL effector repeat sequences; d) assembling the at least one first expression vector and the at least one second expression vector with a third destination vector in a second reaction mixture by said acoustic process; and e) incorporating the first TAL effector repeat unit and the second TAL effector repeat unit from the at least one first expression vector and the at least one second expression vector into the third destination vector by said nucleic acid incorporation process to generate the transcription activator-like (TAL) effector endonuclease monomer.

The transcription activator-like (TAL) effector endonuclease monomer can comprise a FokI endonuclease domain, an N-cap and a C-cap. The transcription activator-like (TAL) effector endonuclease monomer can comprise a C-terminal half-repeat. The C-terminal half-repeat can comprise about 15, 20, 21, 22, 23, 24, 25, 26, 27, 28, 29, 30, 35, or 40 amino acid residues.

The plurality of TAL effector repeat modules (or sequences) can comprise at least 1, 2, 3, 4, 5, 6, 7, 8, 9, 10, 11, 12, 13, 14, 15, 16, 17, 18, 19, 20, 21, 22, 23, 24, 25, 30, or more TAL effector repeat modules (or sequences). In some cases, the plurality of TAL effector repeat modules (or sequences) can comprise at least 1, 2, 3, 4, 5, 6, 7, 8, 9, 10, 11, 12 or more TAL effector repeat modules (or sequences). In some instances, the plurality of TAL effector repeat modules (or sequences) is a first plurality of TAL effector repeat modules (or sequences). In some cases, the plurality of TAL effector repeat modules (or sequences) can be a second plurality of TAL effector repeat modules (or sequences).

Each of the plurality of TAL effector repeat modules (or sequences) can comprise a repeat variable-diresidue (RVD). In some cases, a repeat variable-diresidue (RVD) can comprise HD, NG, NI, NK, or NH. In some cases, a transcription activator-like (TAL) effector endonuclease monomer can comprise a RVD that recognizes T at the N-terminal portion of the TAL effector repeat modules (or sequences), at the C-terminal portion of the TAL effector repeat modules (or sequences), or at both termini. In some cases, the insertion of TAL effector repeat modules (or sequences) can remove a LacZ portion of the second vector.

Each TAL effector repeat sequence unit can comprise at least 1, 2, 3, 4, 5, 6, 7, 8, 9, 10, 11, 12, or more TAL effector repeat modules (or sequences). Each TAL effector repeat sequence unit can comprise at least 2 or more TAL effector repeat modules (or sequences). Each TAL effector repeat sequence unit can comprise at least 3 or more TAL effector repeat modules (or sequences). Each TAL effector repeat sequence unit can comprise at least 4 or more TAL effector repeat modules (or sequences). Each TAL effector repeat sequence unit can comprise at least 5 or more TAL effector repeat modules (or sequences). Each TAL effector repeat sequence unit can comprise at least 6 or more TAL effector repeat modules (or sequences). Each TAL effector repeat sequence unit can comprise at least 7 or more TAL effector repeat modules (or sequences). Each TAL effector repeat sequence unit can comprise at least 8 or more TAL effector repeat modules (or sequences). Each TAL effector repeat sequence unit can comprise at least 9 or more TAL effector repeat modules (or sequences). Each TAL effector repeat sequence unit can comprise at least 10 or more TAL effector repeat modules (or sequences). In some cases, the TAL effector repeat sequence unit can be a first TAL effector repeat sequence unit. In some cases, the TAL effector repeat sequence unit can be a second TAL effector repeat sequence unit.

In some cases, a restriction enzyme is added to a reaction mixture to remove an empty vector or a vector that has not incorporated a polynucleotide of interest. In some cases, the restriction enzyme is a first restriction enzyme, utilized in a first reaction mixture. In some cases, the restriction enzyme is a second restriction enzyme, utilized in a second reaction mixture. In some cases, the restriction enzyme is BsaI or BsaI-HF.

In some cases, the first reaction mixture can further comprise a deoxyribonuclease (DNase). A deoxyribonuclease used herein can cut at an internal site within the DNA. A deoxyribonuclease used herein can target a linear plasmid, thereby removing a non-ligated plasmid. In some cases, a deoxyribonuclease used herein can be Plasmid Safe DNase (Epicentre).

In some instances, the deoxyribonuclease and/or the restriction enzyme (e.g., BsaI or BsaI-HF) can be incubated in the reaction mixture (e.g., a first reaction mixture) for at least 30 minutes, at least 40 minutes, at least 50 minutes, at least 60 minutes, at least 70 minutes, at least 80 minutes, at least 90 minutes, at least 2 hours, at least 3 hours, at least 4 hours, at least 5 hours, at least 6 hours, at least 10 hours, at least 12 hours, or more. The incubation temperature can be about 37° C.

In some cases, the deoxyribonuclease and/or the restriction enzyme (e.g., BsaI or BsaI-HF) can be incubated in a first reaction mixture for at least 30 minutes, at least 40 minutes, at least 50 minutes, at least 60 minutes, at least 70 minutes, at least 80 minutes, at least 90 minutes, at least 2 hours, at least 3 hours, at least 4 hours, at least 5 hours, at least 6 hours, at least 10 hours, at least 12 hours, or more. The incubation temperature can be about 37° C.

In other cases, the deoxyribonuclease and/or the restriction enzyme (e.g., BsaI or BsaI-HF) can be incubated in a second reaction mixture for at least 30 minutes, at least 40 minutes, at least 50 minutes, at least 60 minutes, at least 70 minutes, at least 80 minutes, at least 90 minutes, at least 2 hours, at least 3 hours, at least 4 hours, at least 5 hours, at least 6 hours, at least 10 hours, at least 12 hours, or more. The incubation temperature can be about 37° C.

Upon incubation with the deoxyribonuclease and/or the restriction enzyme (e.g., BsaI or BsaI-HF), the reaction mixture (e.g., a first reaction mixture or a second reaction mixture) can further undergo a transformation step, a culturing step and a plasmid harvesting step. A plasmid obtained from the plasmid harvesting step can further be quantified by a spectrophotometric method, such as by measurement of DNA concentration at UV 280 nm.

A nucleic acid incorporation process described herein can comprise at least one round of a digestion step and a ligation step. The nucleic acid incorporation process can comprise about 2, 3, 4, 5, 6, 7, 8, 9, 10, 15, 20, or more rounds of a digestion step and a ligation step. In some cases, the digestion step is at about 37° C. In some instances, the ligation step is at about 16° C. The time for the digestion step can be at least 2, 3, 4, 5, 6, 7, 8, 9, 10, 15, 30, or more minutes per round. The time for the ligation step can be about 5, 6, 7, 8, 9, 10, 15, 30, 45, 60, or more minutes per round.

The nucleic acid incorporation process can further comprise a background reduction step. The background reduction step can occur after at least one round of a digestion step and a ligation step. The background reduction step can occur at a temperature of about 45° C., 50° C., 55° C., 60° C., or higher. The time for the background reduction step can be about 5, 10, 15, 20, or more minutes.

The nucleic acid incorporation process can further comprise a heat inactivation step. The heat inactivation step can occur at a temperature of about 65° C., 70° C., 75° C., 80° C., 85° C., 90° C., or higher. The time for the heat inactivation step can be about 5, 10, 15, 20, or more minutes.

The first vector can be a destination vector. The first vector can be pFUS vector. The first vector can be pUC18. Alternatively, the first vector can be pUC19.

The second vector can be a destination vector. The second vector can be pFUS vector. The second vector can be pUC18. The second vector can be pUC19.

The third vector can be a destination vector. In some cases, the third vector further comprises a polynucleotide encoding a C-terminal half-repeat, a polynucleotide encoding FokI, a polynucleotide encoding a linker region or a combination thereof. In some cases, the third vector can be pVax vector. The pVax vector can further comprise polynucleotide encoding a C-terminal half-repeat, a polynucleotide encoding FokI, a polynucleotide encoding a linker region or a combination thereof.

In some cases, the volume of a reaction mixture is less than about 10 μL. The volume of a reaction mixture can be less than about 9 μL, less than about 8 μL, less than about 7 μL, less than about 6 μL, less than about 5 μL, less than about 4 μL, less than about 3 μL, less than about 2 μL, or less than about 1 μL. The volume of a reaction mixture can be about 10 μL, about 9 μL, about 8 μL, about 7 μL, about 6 μL, about 5 μL, about 4 μL, about 3 μL, about 2 μL, about 1 μL, or about 0.5 μL. The volume of a reaction mixture can be about 10 μL. The volume of a reaction mixture can be about 5 μL. The volume of a reaction mixture can be about 4 μL. The volume of a reaction mixture can be about 3 μL. The volume of a reaction mixture can be about 2 μL. The volume of a reaction mixture can be about 1 μL. The volume of a reaction mixture can be about 0.5 μL. The reaction mixture can be a first reaction mixture. The reaction mixture can be a second reaction mixture.

In some instances, after treatment of the reaction mixture by a digestion and ligation step, the treated reaction mixture is utilized to transform a production cell for amplification of a TAL product from the reaction mixture. In some instances, the transformed cell is further cultured in media (e.g., LB media) for up to 20-24 hours at a temperature of from about 20° C. to about 37° C. In some cases, the transformed cell is grown in a culture media at a volume of about 1 mL, 2 mL, 3 mL, 4 mL, 5 mL, or more. In some cases, the transformed cell is grown in a cultured media without a prior step of plating onto an agar plate.

The acoustic process can be generated by a high-throughput acoustic liquid handler instrument, such as a Labcyte Echo 550.

### Zinc Finger Nuclease Polypeptide

Similar to TALEN, zinc-finger nuclease (ZFN) is a restriction enzyme that can be engineered to target and edit specific nucleic acid sequences. A ZFN can comprise a zinc-finger DNA binding domain linked either directly or indirectly to a nuclease domain. The zinc-finger DNA binding domain can comprise a set of zinc finger motifs. Each zinc finger motif can be about 30 amino acids in length and can fold into a ββα structure in which the α-helix can be inserted into the major groove of the DNA double helix and can engage in sequence-specific interaction with the DNA site. In some cases, the sequence-specific recognition can span over 3 base pairs. In some cases, a single zinc finger motif can interact specifically with 1, 2 or 3 nucleotides.

A zinc-finger DNA binding domain of a ZFN can comprise from about 1 to about 10 zinc finger motifs. A zinc-finger DNA binding domain can comprise from about 1 to about 9, from about 2 to about 8, from about 2 to about 6 or from about 2 to about 4 zinc finger motifs. In some cases, a zinc-finger DNA binding domain can comprise at least 1, 2, 3, 4, 5, 6, 7, 8, 9, 10, or more zinc finger motifs. A zinc-finger DNA binding domain can comprise at least 1, 2, 3, 4, 5, 6, 7, 8, 9, or 10 zinc finger motifs. A zinc-finger DNA binding domain can comprise about 1 zinc finger motif. A zinc-finger DNA binding domain can comprise about 2 zinc finger motif. A zinc-finger DNA binding domain can comprise about 3 zinc finger motif. A zinc-finger DNA binding domain can comprise about 4 zinc finger motif. A zinc-finger DNA binding domain can comprise about 5 zinc finger motif. A zinc-finger DNA binding domain can comprise about 6 zinc finger motif. A zinc-finger DNA binding domain can comprise about 7 zinc finger motif. A zinc-finger DNA binding domain can comprise about 8 zinc finger motif. A zinc-finger DNA binding domain can comprise about 9 zinc finger motif. A zinc-finger DNA binding domain can comprise about 10 zinc finger motif.

A zinc finger motif can be a wild-type zinc finger motif or a modified zinc finger motif enhanced for specific recognition of a set of nucleotides. A ZFN described herein can comprise one or more wild-type zinc finger motif. A ZFN described herein can comprise one or more modified zinc finger motif enhanced for specific recognition of a set of nucleotides. A modified zinc finger motif can comprise 1, 2, 3, 4, 5, 6, 7, 8, 9, 10, 15, 20, 25, or more mutations that can enhance the motif for specific recognition of a set of nucleotides. In some cases, one or more amino acid residues within the α-helix of a zinc finger motif are modified. In some cases, one or more amino acid residues at positions −1, +1, +2, +3, +4, +5, and/or +6 relative to the N-terminus of the α-helix of a zinc finger motif can be modified.

A nuclease domain linked to a zinc-finger DNA-binding domain can be an endonuclease or an exonuclease. An endonuclease can include restriction endonucleases and homing endonucleases. An endonuclease can also include S1 Nuclease, mung bean nuclease, pancreatic DNase I, micrococcal nuclease, or yeast HO endonuclease. An exonuclease can include a 3′-5′ exonuclease or a 5′-3′ exonuclease. An exonuclease can also include a DNA exonuclease or an RNA exonuclease. Examples of exonuclease includes exonucleases I, II, III, IV, V and VIII; DNA polymerase I, RNA exonuclease 2, and the like.

A nuclease domain fused to a zinc-finger DNA-binding domain can be a restriction endonuclease (or restriction enzyme). In some instances, a restriction enzyme cleaves DNA at a site removed from the recognition site and has a separate binding and cleavage domains. In some instances, such restriction enzyme is a Type IIS restriction enzyme.

A nuclease domain fused to a zinc-finger DNA-binding domain can be a Type IIS nuclease. A Type IIS nuclease can be FokI or Bfil. In some cases, a nuclease domain fused to a zinc-finger DNA-binding domain is FokI. In other cases, a nuclease domain fused to a zinc-finger DNA-binding domain is Bfil.

FokI can be a wild-type FokI or can comprise one or more mutations. In some cases, FokI can comprise about 1, 2, 3, 4, 5, 6, 7, 8, 9, 10, or more mutations. A mutation can enhance cleavage efficiency. A mutation can abolish cleavage activity. In some cases, a mutation can enhance homodimerization. For example, FokI can have a mutation at one or more amino acid residue positions 446, 447, 479, 483, 484, 486, 487, 490, 491, 496, 498, 499, 500, 531, 534, 537, and 538 to modulate homodimerization.

In some instances, a FokI cleavage domain is, for example, as described in Kim et al. “Hybrid restriction enzymes: Zinc finger fusions to Fok I cleavage domain,” PNAS 93: 1156-1160 (1996), which is incorporated herein by reference in its entirety. In some cases, a FokI cleavage domain described herein is a FokI of SEQ ID NO: 1 (Table 5). In other instances, a FokI cleavage domain described herein is a FokI, for example, as described in U.S. Pat. No. 8,586,526, which is incorporated herein by reference in its entirety.

A nuclease domain can be linked to a zinc-finger DNA-binding domain either directly or through a linker. A linker can be between about 1 to about 50 amino acid residues in length. A linker can be from about 5 to about 45, from about 5 to about 40, from about 5 to about 35, from about 5 to about 30, from about 5 to about 25, from about 5 to about 20, from about 5 to about 15, from about 10 to about 40, from about 10 to about 35, from about 10 to about 30, from about 10 to about 25, from about 10 to about 20, from about 12 to about 40, from about 12 to about 35, from about 12 to about 30, from about 12 to about 25, from about 12 to about 20, from about 14 to about 40, from about 14 to about 35, from about 14 to about 30, from about 14 to about 25, from about 14 to about 20, from about 14 to about 16, from about 15 to about 40, from about 15 to about 35, from about 15 to about 30, from about 15 to about 25, from about 15 to about 20, from about 15 to about 18, from about 18 to about 40, from about 18 to about 35, from about 18 to about 30, from about 18 to about 25, from about 18 to about 24, from about 20 to about 40, from about 20 to about 35, from about 20 to about 30, or from about 25 to about 30 amino acid residues in length.

A linker for linking a nuclease domain to a zinc-finger DNA-binding domain can be about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, or 50 amino acid residues in length. A linker can be about 10 amino acid residues in length. A linker can be about 11 amino acid residues in length. A linker can be about 12 amino acid residues in length. A linker can be about 13 amino acid residues in length. A linker can be about 14 amino acid residues in length. A linker can be about 15 amino acid residues in length. A linker can be about 16 amino acid residues in length. A linker can be about 17 amino acid residues in length. A linker can be about 18 amino acid residues in length. A linker can be about 19 amino acid residues in length. A linker can be about 20 amino acid residues in length. A linker can be about 21 amino acid residues in length. A linker can be about 22 amino acid residues in length. A linker can be about 23 amino acid residues in length. A linker can be about 24 amino acid residues in length. A linker can be about 25 amino acid residues in length. A linker can be about 26 amino acid residues in length. A linker can be about 27 amino acid residues in length. A linker can be about 28 amino acid residues in length. A linker can be about 29 amino acid residues in length. A linker can be about 30 amino acid residues in length.

### Methods of Generating a ZFN

In some instances, a method of generating a zinc-finger nuclease monomer is provided herein. A method of generating a ZFN monomer can comprise the steps of (a) assembling a first plurality of zinc-finger motif sequences in a first reaction mixture comprising a plurality of first destination vectors; (b) incorporating the first plurality of zinc-finger motif sequences into at least one first destination vector from the plurality of first destination vectors by a nucleic acid incorporation process to generate at least one first expression vector, wherein the at least one first expression vector comprises a first zinc-finger repeat unit and wherein the first zinc-finger repeat unit comprises the first plurality of zinc-finger motif sequences; (c) incubating the first reaction mixture comprising the at least one first expression vector from step b) with a first restriction enzyme to remove a first destination vector that fails to incorporate the first plurality of zinc-finger motif sequences; (d) repeating steps a) to c) with a second plurality of zinc-finger motif sequences and a plurality of second destination vectors to generate at least one second expression vector, wherein the at least one second expression vector comprises a second zinc-finger repeat unit and wherein the second zinc-finger repeat unit comprises the second plurality of zinc-finger motif sequences; (e) assembling the at least one first expression vector and the at least one second expression vector with a third destination vector in a second reaction mixture; and (f) incorporating the first zinc-finger repeat unit and the second zinc-finger repeat unit from the at least one first expression vector and the at least one second expression vector into the third destination vector by said nucleic acid incorporation process to generate the nucleic acid construct containing the ZFN monomer.

In some cases, a method of generating a ZFN monomer can comprise the step of a) assembling a first plurality of zinc-finger motif sequences and a plurality of first destination vectors in a first reaction mixture by an acoustic process; b) incorporating the first plurality of zinc-finger motif sequences into at least one first destination vector from the plurality of first destination vectors by a nucleic acid incorporation process to generate at least one first expression vector, wherein the at least one first expression vector comprises a first zinc-finger repeat unit and wherein the first zinc-finger repeat unit comprises the first plurality of zinc-finger motif sequences; c) repeating steps a) and b) with a second plurality of zinc-finger motif sequences and a plurality of second destination vectors to generate at least one second expression vector, wherein the at least one second expression vector comprises a second zinc-finger repeat unit and wherein the second zinc-finger repeat unit comprises the second plurality of zinc-finger motif sequences; d) assembling the at least one first expression vector and the at least one second expression vector with a third destination vector in a second reaction mixture by said acoustic process; and e) incorporating the first zinc-finger repeat unit and the second zinc-finger repeat unit from the at least one first expression vector and the at least one second expression vector into the third destination vector by said nucleic acid incorporation process to generate the ZFN monomer.

The plurality of zinc-finger repeat sequences can comprise at least 1, 2, 3, 4, 5, 6, 7, 8, 9, or 10 zinc-finger repeat sequences. The plurality of zinc-finger repeat sequences can comprise at least 2 zinc-finger repeat sequences. The plurality of zinc-finger repeat sequences can comprise at least 3 zinc-finger repeat sequences. The plurality of zinc-finger repeat sequences can comprise at least 4 zinc-finger repeat sequences. The plurality of zinc-finger repeat sequences can comprise at least 5 zinc-finger repeat sequences. The plurality of zinc-finger repeat sequences can comprise at least 6 zinc-finger repeat sequences. The plurality of zinc-finger repeat sequences can comprise at least 7 zinc-finger repeat sequences. The plurality of zinc-finger repeat sequences can comprise at least 8 zinc-finger repeat sequences. The plurality of zinc-finger repeat sequences can comprise at least 9 zinc-finger repeat sequences. The plurality of zinc-finger repeat sequences can comprise at least 10 zinc-finger repeat sequences. In some cases, the plurality of zinc-finger repeat sequences can be a first plurality of zinc-finger repeat sequences. Other times, the plurality of zinc-finger repeat sequences can be a second plurality of zinc-finger repeat sequences.

In some cases, a restriction enzyme is added to a reaction mixture to remove an empty vector or a vector that has not incorporated a polynucleotide of interest. In some cases, the restriction enzyme is a first restriction enzyme, utilized in a first reaction mixture. In some cases, the restriction enzyme is a second restriction enzyme, utilized in a second reaction mixture. In some cases, the restriction enzyme is BsaI or BsaI-HF.

In some cases, the first reaction mixture can further comprise a deoxyribonuclease (DNase). A deoxyribonuclease used herein can cut at an internal site within the DNA. A deoxyribonuclease used herein can target a linear plasmid, thereby removing a non-ligated plasmid. In some cases, a deoxyribonuclease used herein can be Plasmid Safe DNase (Epicentre).

In some instances, the deoxyribonuclease and/or the restriction enzyme (e.g., BsaI or BsaI-HF) can be incubated in the reaction mixture (e.g., a first reaction mixture) for at least 30 minutes, at least 40 minutes, at least 50 minutes, at least 60 minutes, at least 70 minutes, at least 80 minutes, at least 90 minutes, at least 2 hours, at least 3 hours, at least 4 hours, at least 5 hours, at least 6 hours, at least 10 hours, at least 12 hours, or more. The incubation temperature can be about 37° C.

In some cases, the deoxyribonuclease and/or the restriction enzyme (e.g., BsaI or BsaI-HF) can be incubated in a first reaction mixture for at least 30 minutes, at least 40 minutes, at least 50 minutes, at least 60 minutes, at least 70 minutes, at least 80 minutes, at least 90 minutes, at least 2 hours, at least 3 hours, at least 4 hours, at least 5 hours, at least 6 hours, at least 10 hours, at least 12 hours, or more. The incubation temperature can be about 37° C.

In other cases, the deoxyribonuclease and/or the restriction enzyme (e.g., BsaI or BsaI-HF) can be incubated in a second reaction mixture for at least 30 minutes, at least 40 minutes, at least 50 minutes, at least 60 minutes, at least 70 minutes, at least 80 minutes, at least 90 minutes, at least 2 hours, at least 3 hours, at least 4 hours, at least 5 hours, at least 6 hours, at least 10 hours, at least 12 hours, or more. The incubation temperature can be about 37° C.

Upon incubation with the deoxyribonuclease and/or the restriction enzyme (e.g., BsaI or BsaI-HF), the reaction mixture (e.g., a first reaction mixture or a second reaction mixture) can further undergo a transformation step, a culturing step and a plasmid harvesting step. A plasmid obtained from the plasmid harvesting step can further be quantified by a spectrophotometric method, such as by measurement of DNA concentration at UV 280 nm.

A nucleic acid incorporation process described herein can comprise at least one round of a digestion step and a ligation step. The nucleic acid incorporation process can comprise about 2, 3, 4, 5, 6, 7, 8, 9, 10, 15, 20, or more rounds of a digestion step and a ligation step. In some cases, the digestion step is at about 37° C. In some instances, the ligation step is at about 16° C. The time for the digestion step can be at least 2, 3, 4, 5, 6, 7, 8, 9, 10, 15, 30, or more minutes per round. The time for the ligation step can be about 5, 6, 7, 8, 9, 10, 15, 30, 45, 60, or more minutes per round.

The nucleic acid incorporation process can further comprise a background reduction step. The background reduction step can occur after at least one round of a digestion step and a ligation step. The background reduction step can occur at a temperature of about 45° C., 50° C., 55° C., 60° C., or higher. The time for the background reduction step can be about 5, 10, 15, 20, or more minutes.

The nucleic acid incorporation process can further comprise a heat inactivation step. The heat inactivation step can occur at a temperature of about 65° C., 70° C., 75° C., 80° C., 85° C., 90° C., or higher. The time for the heat inactivation step can be about 5, 10, 15, 20, or more minutes.

The first vector can be a destination vector. The first vector can be pFUS vector. The first vector can be pUC18. Alternatively, the first vector can be pUC19.

The second vector can be a destination vector. The second vector can be pFUS vector. The second vector can be pUC18. The second vector can be pUC19.

The third vector can be a destination vector. In some cases, the third vector further comprises a polynucleotide encoding FokI, a polynucleotide encoding a linker region or a combination thereof. In some cases, the third vector can be pVax vector. The pVax vector can further comprise a polynucleotide encoding FokI, a polynucleotide encoding a linker region or a combination thereof.

In some cases, the volume of a reaction mixture is less than about 10 μL. The volume of a reaction mixture can be less than about 9 μL, less than about 8 μL, less than about 7 μL, less than about 6 μL, less than about 5 μL, less than about 4 μL, less than about 3 μL, less than about 2 μL or less than about 1 μL. The volume of a reaction mixture can be about 10 μL, about 9 μL, about 8 μL, about 7 μL, about 6 μL, about 5 μL, about 4 μL, about 3 μL, about 2 μL, about 1 μL or about 0.5 μL. The volume of a reaction mixture can be about 10 μL. The volume of a reaction mixture can be about 5 μL. The volume of a reaction mixture can be about 4 μL. The volume of a reaction mixture can be about 3 μL. The volume of a reaction mixture can be about 2 μL. The volume of a reaction mixture can be about 1 μL. The volume of a reaction mixture can be about 0.5 μL. The reaction mixture can be a first reaction mixture. The reaction mixture can be a second reaction mixture.

In some instances, after treatment of the reaction mixture by a digestion and ligation step, the treated reaction mixture is utilized to transform a production cell for amplification of a ZFN product from the reaction mixture. In some instances, the transformed cell is further cultured in media (e.g., LB media) for up to 20-24 hours at a temperature of from about 20° C. to about 37° C. In some cases, the transformed cell is grown in a culture media at a volume of about 1 mL, 2 mL, 3 mL, 4 mL, 5 mL, or more. In some cases, the transformed cell is grown in a cultured media without a prior step of plating onto an agar plate.

The acoustic process can be generated by a high-throughput acoustic liquid handler instrument, such as a Labcyte Echo 550.

### Additional Polypeptides of Interest

In additional cases, a plurality of polynucleotides of interest comprises polynucleotides that encode one or more fusion polypeptides or a protein of interest. A protein of interest can be an eukaryotic protein or a prokaryotic protein. A protein of interest can be an enzyme, a transporter, a receptor, a channel protein, an adaptor protein, a chaperone, a signaling protein, a plasma protein, transcription related protein, translation related protein, mitochondrial protein, or cytoskeleton related protein. As used herein, the term “protein” or “protein of interest” can also include its functional fragment thereof.

In some instances, provided herein is a method of generating a protein of interest. A method of generating a protein of interest can comprise the step of (a) assembling a first plurality of polynucleotides of interest in a first reaction mixture comprising a plurality of first destination vectors; (b) incorporating the first plurality of polynucleotides of interest into at least one first destination vector from the plurality of first destination vectors by a nucleic acid incorporation process to generate at least one first expression vector, wherein the at least one first expression vector comprises a first polynucleotide unit and wherein the first polynucleotide unit comprises the first plurality of polynucleotides of interest; (c) incubating the first reaction mixture comprising the at least one first expression vector from step b) with a first restriction enzyme to remove a first destination vector that fails to incorporate the first plurality of polynucleotides of interest; (d) repeating steps a) to c) with a second plurality of polynucleotides of interest and a plurality of second destination vectors to generate at least one second expression vector, wherein the at least one second expression vector comprises a second polynucleotide unit and wherein the second polynucleotide unit comprises the second plurality of polynucleotides of interest; (e) assembling the at least one first expression vector and the at least one second expression vector with a third destination vector in a second reaction mixture; and (f) incorporating the first polynucleotide unit and the second polynucleotide unit from the at least one first expression vector and the at least one second expression vector into the third destination vector by said nucleic acid incorporation process to generate the nucleic acid construct containing a plurality of polynucleotides of interest.

In some cases, a method of generating a protein of interest can comprise the step of (a) assembling a first plurality of polynucleotides of interest and a plurality of first destination vectors in a first reaction mixture by an acoustic process; (b) incorporating the first plurality of polynucleotides of interest into at least one first destination vector from the plurality of first destination vectors by a nucleic acid incorporation process to generate at least one first expression vector, wherein the at least one first expression vector comprises a first polynucleotide unit and wherein the first polynucleotide unit comprises the first plurality of polynucleotides of interest; (c) repeating steps a) and b) with a second plurality of polynucleotides of interest and a plurality of second destination vectors to generate at least one second expression vector, wherein the at least one second expression vector comprises a second polynucleotide unit and wherein the second polynucleotide unit comprises the second plurality of polynucleotides of interest; (d) assembling the at least one first expression vector and the at least one second expression vector with a third destination vector in a second reaction mixture by said acoustic process; and (e) incorporating the first polynucleotide unit and the second polynucleotide unit from the at least one first expression vector and the at least one second expression vector into the third destination vector by said nucleic acid incorporation process to generate the nucleic acid construct containing a plurality of polynucleotides of interest.

A plurality of polynucleotide of interest can comprise at least 1, 2, 3, 4, 5, 6, 7, 8, 9, 10, 15, 20, or more polynucleotide modules, in which each of the polynucleotide module comprise a portion of the polynucleotide of interest. A plurality of polynucleotide of interest can comprise at least 2 or more polynucleotide modules, in which each of the polynucleotide module comprise a portion of the polynucleotide of interest. A plurality of polynucleotide of interest can comprise at least 3 or more polynucleotide modules, in which each of the polynucleotide module comprise a portion of the polynucleotide of interest. A plurality of polynucleotide of interest can comprise at least 4 or more polynucleotide modules, in which each of the polynucleotide module comprise a portion of the polynucleotide of interest. A plurality of polynucleotide of interest can comprise at least 5 or more polynucleotide modules, in which each of the polynucleotide module comprise a portion of the polynucleotide of interest. A plurality of polynucleotide of interest can comprise at least 6 or more polynucleotide modules, in which each of the polynucleotide module comprise a portion of the polynucleotide of interest. A plurality of polynucleotide of interest can comprise at least 7 or more polynucleotide modules, in which each of the polynucleotide module comprise a portion of the polynucleotide of interest. A plurality of polynucleotide of interest can comprise at least 8 or more polynucleotide modules, in which each of the polynucleotide module comprise a portion of the polynucleotide of interest. A plurality of polynucleotide of interest can comprise at least 9 or more polynucleotide modules, in which each of the polynucleotide module comprise a portion of the polynucleotide of interest. A plurality of polynucleotide of interest can comprise at least 10 or more polynucleotide modules, in which each of the polynucleotide module comprise a portion of the polynucleotide of interest. A plurality of polynucleotide of interest can comprise at least 15 or more polynucleotide modules, in which each of the polynucleotide module comprise a portion of the polynucleotide of interest. A plurality of polynucleotide of interest can comprise at least 20 or more polynucleotide modules, in which each of the polynucleotide module comprise a portion of the polynucleotide of interest. A plurality of polynucleotide of interest can be a first plurality of polynucleotide of interest. A plurality of polynucleotide of interest can be a second plurality of polynucleotide of interest.

In some cases, a restriction enzyme is added to a reaction mixture to remove an empty vector or a vector that has not incorporated a polynucleotide of interest. In some cases, the restriction enzyme is a first restriction enzyme, utilized in a first reaction mixture. In some cases, the restriction enzyme is a second restriction enzyme, utilized in a second reaction mixture. In some cases, the restriction enzyme is BsaI or BsaI-HF.

In some cases, the first reaction mixture can further comprise a deoxyribonuclease (DNase). A deoxyribonuclease used herein can cut at an internal site within the DNA. A deoxyribonuclease used herein can target a linear plasmid, thereby removing a non-ligated plasmid. In some cases, a deoxyribonuclease used herein can be Plasmid Safe DNase (Epicentre).

In some instances, the deoxyribonuclease and/or the restriction enzyme (e.g., BsaI or BsaI-HF) can be incubated in the reaction mixture for at least 30 minutes, at least 40 minutes, at least 50 minutes, at least 60 minutes, at least 70 minutes, at least 80 minutes, at least 90 minutes, at least 2 hours, at least 3 hours, at least 4 hours, at least 5 hours, at least 6 hours, at least 10 hours, at least 12 hours, or more. The incubation temperature can be about 37° C.

In some cases, the deoxyribonuclease and/or the restriction enzyme (e.g., BsaI or BsaI-HF) can be incubated in a first reaction mixture for at least 30 minutes, at least 40 minutes, at least 50 minutes, at least 60 minutes, at least 70 minutes, at least 80 minutes, at least 90 minutes, at least 2 hours, at least 3 hours, at least 4 hours, at least 5 hours, at least 6 hours, at least 10 hours, at least 12 hours, or more. The incubation temperature can be about 37° C.

In other cases, the deoxyribonuclease and/or the restriction enzyme (e.g., BsaI or BsaI-HF) can be incubated in a second reaction mixture for at least 30 minutes, at least 40 minutes, at least 50 minutes, at least 60 minutes, at least 70 minutes, at least 80 minutes, at least 90 minutes, at least 2 hours, at least 3 hours, at least 4 hours, at least 5 hours, at least 6 hours, at least 10 hours, at least 12 hours, or more. The incubation temperature can be about 37° C.

Upon incubation with the deoxyribonuclease and/or the restriction enzyme (e.g., BsaI or BsaI-HF), the reaction mixture (e.g., a first reaction mixture or a second reaction mixture) can further undergo a transformation step, a culturing step and a plasmid harvesting step. A plasmid obtained from the plasmid harvesting step can further be quantified by a spectrophotometric method, such as by measurement of DNA concentration at UV 280 nm.

A nucleic acid incorporation process described herein can comprise at least one round of a digestion step and a ligation step. The nucleic acid incorporation process can comprise about 2, 3, 4, 5, 6, 7, 8, 9, 10, 15, 20, or more rounds of a digestion step and a ligation step. In some cases, the digestion step is at about 37° C. In some instances, the ligation step is at about 16° C. The time for the digestion step can be at least 2, 3, 4, 5, 6, 7, 8, 9, 10, 15, 30, or more minutes per round. The time for the ligation step can be about 5, 6, 7, 8, 9, 10, 15, 30, 45, 60, or more minutes per round.

The nucleic acid incorporation process can further comprise a background reduction step. The background reduction step can occur after at least one round of a digestion step and a ligation step. The background reduction step can occur at a temperature of about 45° C., 50° C., 55° C., 60° C., or higher. The time for the background reduction step can be about 5, 10, 15, 20, or more minutes.

The nucleic acid incorporation process can further comprise a heat inactivation step. The heat inactivation step can occur at a temperature of about 65° C., 70° C., 75° C., 80° C., 85° C., 90° C., or higher. The time for the heat inactivation step can be about 5, 10, 15, 20, or more minutes.

The first vector can be a destination vector. The first vector can be pFUS vector. The first vector can be pUC18. Alternatively, the first vector can be pUC19.

The second vector can be a destination vector. The second vector can be pFUS vector. The second vector can be pUC18. The second vector can be pUC19.

The third vector can be a destination vector. In some cases, the third vector can be pVax vector.

In some cases, the volume of a reaction mixture is less than about 10 μL. The volume of a reaction mixture can be less than about 9 μL, less than about 8 μL, less than about 7 μL, less than about 6 μL, less than about 5 μL, less than about 4 μL, less than about 3 μL, less than about 2 μL or less than about 1 μL. The volume of a reaction mixture can be about 10 μL, about 9 μL, about 8 μL, about 7 μL, about 6 μL, about 5 μL, about 4 μL, about 3 μL, about 2 μL, about 1 μL or about 0.5 μL. The volume of a reaction mixture can be about 10 μL. The volume of a reaction mixture can be about 5 μL. The volume of a reaction mixture can be about 4 μL. The volume of a reaction mixture can be about 3 μL. The volume of a reaction mixture can be about 2 μL. The volume of a reaction mixture can be about 1 μL. The volume of a reaction mixture can be about 0.5 μL. The reaction mixture can be a first reaction mixture. The reaction mixture can be a second reaction mixture.

The acoustic process can be generated by a high-throughput acoustic liquid handler instrument, such as a Labcyte Echo 550.

### Targets

In some aspects, described herein include methods of modifying the genetic material of a target cell utilizing one or more of a polypeptide of interest (e.g., a TALEN or a ZFN) described herein. A target cell can be a eukaryotic cell or a prokaryotic cell. A target cell can be an animal cell or a plant cell. An animal cell can include a cell from a marine invertebrate, fish, insects, amphibian, reptile, or mammal. A mammalian cell can be obtained from a primate, ape, equine, bovine, porcine, canine, feline, or rodent. A mammal can be a primate, ape, dog, cat, rabbit, ferret, or the like. A rodent can be a mouse, rat, hamster, gerbil, hamster, chinchilla, or guinea pig. A bird cell can be from a canary, parakeet or parrots. A reptile cell can be from a turtles, lizard or snake. A fish cell can be from a tropical fish. For example, the fish cell can be from a zebrafish (e.g., Danino rerio). A worm cell can be from a nematode (e.g., C. elegans). An amphibian cell can be from a frog. An arthropod cell can be from a tarantula or hermit crab.

A mammalian cell can also include cells obtained from a primate (e.g., a human or a non-human primate). A mammalian cell can include an epithelial cell, connective tissue cell, hormone secreting cell, a nerve cell, a skeletal muscle cell, a blood cell, an immune system cell, or a stem cell.

Exemplary mammalian cells can include, but are not limited to, 293A cell line, 293FT cell line, 293F cells, 293 H cells, HEK 293 cells, CHO DG44 cells, CHO-S cells, CHO-Kl cells, Expi293F™ cells, Flp-In™ T-REx™ 293 cell line, Flp-In™-293 cell line, Flp-In™-3T3 cell line, Flp-In™-BHK cell line, Flp-In™-CHO cell line, Flp-In™-CV-1 cell line, Flp-In™-Jurkat cell line, FreeStyle™ 293-F cells, FreeStyle™ CHO-S cells, GripTite™ 293 MSR cell line, GS-CHO cell line, HepaRG™ cells, T-REx™ Jurkat cell line, Per.C6 cells, T-REx™-293 cell line, T-REx™-CHO cell line, T-REx™-HeLa cell line, NC-HIMT cell line, and PC12 cell line.

In some instances, a target cell is a cell comprising one or more modifications within its genome. For example, a target cell can have one or more insertions, deletions, or mutations within its genome, in which one or more TALENs can target and edit the modification site(s).

In some instances, a target cell is a cell comprising one or more single nucleotide polymorphism (SNP). In some instances, a TALEN described herein is designed to target and edit a target cell comprising a SNP.

In some cases, a target cell is a cell that does not contain a modification. For example, a target cell can comprise a genome without genetic defect (e.g., without genetic mutation) and TALEN described herein can be used to introduce a modification (e.g., a mutation) within the genome.

In some cases, a target cell is a cancerous cell. Cancer can be a solid tumor or a hematologic malignancy. The solid tumor can include a sarcoma or a carcinoma. Exemplary sarcoma target cell can include, but are not limited to, cell obtained from alveolar rhabdomyosarcoma, alveolar soft part sarcoma, ameloblastoma, angiosarcoma, chondrosarcoma, chordoma, clear cell sarcoma of soft tissue, dedifferentiated liposarcoma, desmoid, desmoplastic small round cell tumor, embryonal rhabdomyosarcoma, epithelioid fibrosarcoma, epithelioid hemangioendothelioma, epithelioid sarcoma, esthesioneuroblastoma, Ewing sarcoma, extrarenal rhabdoid tumor, extraskeletal myxoid chondrosarcoma, extraskeletal osteosarcoma, fibrosarcoma, giant cell tumor, hemangiopericytoma, infantile fibrosarcoma, inflammatory myofibroblastic tumor, Kaposi sarcoma, leiomyosarcoma of bone, liposarcoma, liposarcoma of bone, malignant fibrous histiocytoma (MFH), malignant fibrous histiocytoma (MFH) of bone, malignant mesenchymoma, malignant peripheral nerve sheath tumor, mesenchymal chondrosarcoma, myxofibrosarcoma, myxoid liposarcoma, myxoinflammatory fibroblastic sarcoma, neoplasms with perivascular epitheioid cell differentiation, osteosarcoma, parosteal osteosarcoma, neoplasm with perivascular epitheioid cell differentiation, periosteal osteosarcoma, pleomorphic liposarcoma, pleomorphic rhabdomyo sarcoma, PNET/extraskeletal Ewing tumor, rhabdomyosarcoma, round cell liposarcoma, small cell osteosarcoma, solitary fibrous tumor, synovial sarcoma, or telangiectatic osteosarcoma.

Exemplary carcinoma target cell can include, but are not limited to, cell obtained from anal cancer, appendix cancer, bile duct cancer (i.e., cholangiocarcinoma), bladder cancer, brain tumor, breast cancer, cervical cancer, colon cancer, cancer of Unknown Primary (CUP), esophageal cancer, eye cancer, fallopian tube cancer, gastroenterological cancer, kidney cancer, liver cancer, lung cancer, medulloblastoma, melanoma, oral cancer, ovarian cancer, pancreatic cancer, parathyroid disease, penile cancer, pituitary tumor, prostate cancer, rectal cancer, skin cancer, stomach cancer, testicular cancer, throat cancer, thyroid cancer, uterine cancer, vaginal cancer, or vulvar cancer.

Alternatively, the cancerous cell can comprise cells obtained from a hematologic malignancy. Hematologic malignancy can comprise a leukemia, a lymphoma, a myeloma, a non-Hodgkin's lymphoma, or a Hodgkin's lymphoma. In some cases, the hematologic malignancy can be a T-cell based hematologic malignancy. Other times, the hematologic malignancy can be a B-cell based hematologic malignancy. Exemplary B-cell based hematologic malignancy can include, but are not limited to, chronic lymphocytic leukemia (CLL), small lymphocytic lymphoma (SLL), high-risk CLL, a non-CLL/SLL lymphoma, prolymphocytic leukemia (PLL), follicular lymphoma (FL), diffuse large B-cell lymphoma (DLBCL), mantle cell lymphoma (MCL), Waldenstrom's macroglobulinemia, multiple myeloma, extranodal marginal zone B cell lymphoma, nodal marginal zone B cell lymphoma, Burkitt's lymphoma, non-Burkitt high grade B cell lymphoma, primary mediastinal B-cell lymphoma (PMBL), immunoblastic large cell lymphoma, precursor B-lymphoblastic lymphoma, B cell prolymphocytic leukemia, lymphoplasmacytic lymphoma, splenic marginal zone lymphoma, plasma cell myeloma, plasmacytoma, mediastinal (thymic) large B cell lymphoma, intravascular large B cell lymphoma, primary effusion lymphoma, or lymphomatoid granulomatosis. Exemplary T-cell based hematologic malignancy can include, but are not limited to, peripheral T-cell lymphoma not otherwise specified (PTCL-NOS), anaplastic large cell lymphoma, angioimmunoblastic lymphoma, cutaneous T-cell lymphoma, adult T-cell leukemia/lymphoma (ATLL), blastic NK-cell lymphoma, enteropathy-type T-cell lymphoma, hematosplenic gamma-delta T-cell lymphoma, lymphoblastic lymphoma, nasal NK/T-cell lymphomas, or treatment-related T-cell lymphomas.

In some cases, a cell can be a tumor cell line. Exemplary tumor cell line can include, but are not limited to, 600MPE, AU565, BT-20, BT-474, BT-483, BT-549, Evsa-T, Hs578T, MCF-7, MDA-MB-231, SkBr3, T-47D, HeLa, DU145, PC3, LNCaP, A549, H1299, NCI-H460, A2780, SKOV-3/Luc, Neuro2a, RKO, RKO-AS45-1, HT-29, SW1417, SW948, DLD-1, SW480, Capan-1, MC/9, B72.3, B25.2, B6.2, B38.1, DMS 153, SU.86.86, SNU-182, SNU-423, SNU-449, SNU-475, SNU-387, Hs 817.T, LMH, LMH/2A, SNU-398, PLHC-1, HepG2/SF, OCI-Ly1, OCI-Ly2, OCI-Ly3, OCI-Ly4, OCI-Ly6, OCI-Ly7, OCI-Ly10, OCI-Ly18, OCI-Ly19, U2932, DB, HBL-1, RIVA, SUDHL2, TMD8, MEC1, MEC2, 8E5, CCRF-CEM, MOLT-3, TALL-104, AML-193, THP-1, BDCM, HL-60, Jurkat, RPMI 8226, MOLT-4, RS4, K-562, KASUMI-1, Daudi, GA-10, Raji, JeKo-1, NK-92, and Mino.

### Computational Design of TALENs

### TALEN Mechanism

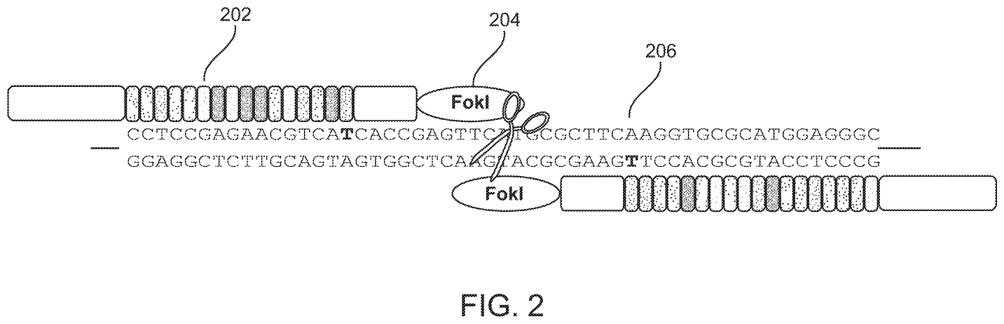

In some aspects, this application also presents a system and related methods that determine candidate residue sequences for transcription activator-like effector nucleases (TALENs) for genome cleavage tasks. FIG. 2 illustrates how TALENs facilitate site-specific DNA sequence cleavage. Transcription activator-like effectors (TALEs) may be transcription activators secreted by plant bacteria Xanthomonas. A TALE has a DNA-binding domain (DBD) 202 that can recognize specific DNA bases, and it is possible to engineer TALEs that specifically bind to a desired DNA sequence 206. An engineered TALE can be fused to a DNA-cutting domain (DCD) 204 or functional cleavage domain, such as FokI, to create a TALEN. Such nucleases can function as a site-specific endonuclease cleaving the target sequence in a genome, which allows various types of genomic engineering, such as gene knockout and gene knock-in. Such nucleases typically function in either homodimer or heterodimer fashion to cleave the DNA within the spacer region, namely the region between the two binding sites. Specifically, two such nucleases can bind to a DNA target in a tail-to-tail orientation (one binding to one strand on one side (e.g., a first side) of a cut site, the other binding to the other strand on the other side of the cut site), as shown in FIG. 2, to allow dimerization of the DNA-cutting domains and generation of a double-stranded break.

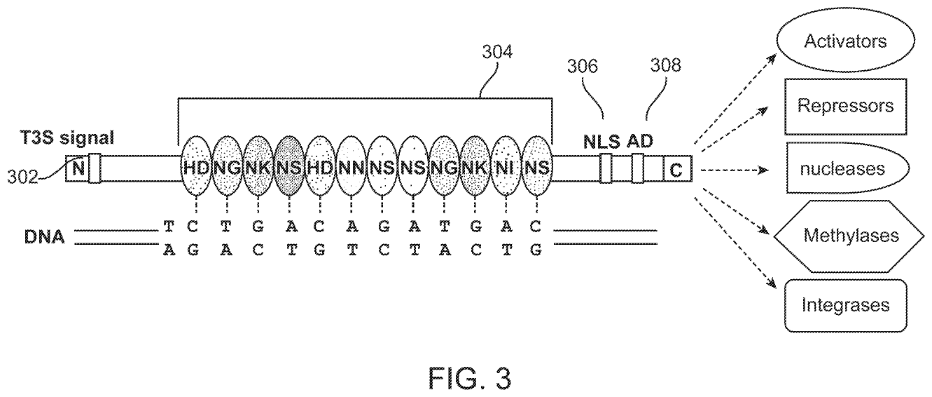

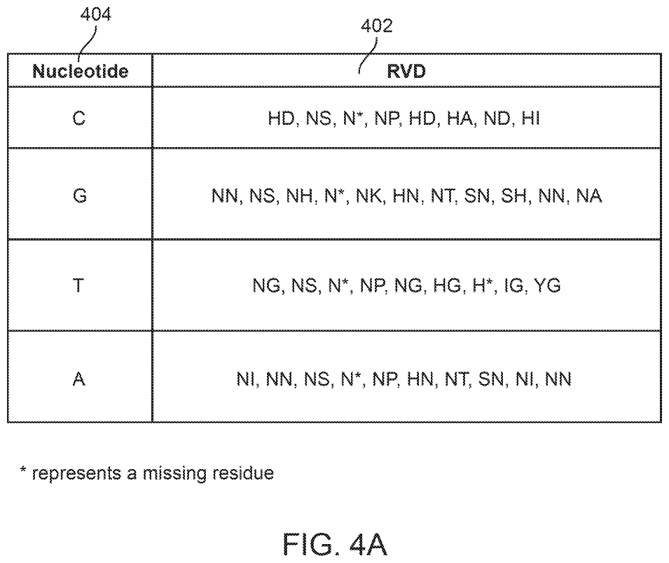

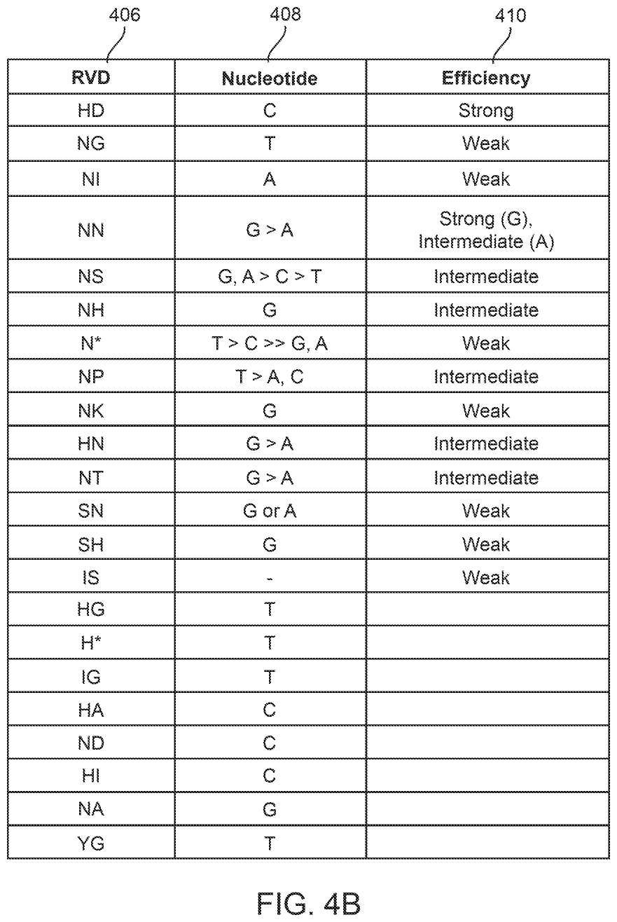

FIG. 3 illustrates the structure of a TALEN. TALEs have specific structural features, including the N-terminal secretion signal 302; a DBD 304 with a variable number of 34/35 amino acid long repeats; a nuclear localization signal 306 and an acidic activation domain at 308 the C-terminus of the protein. The analysis of the TALE structure, in particular their DBD repeats and the sequence of the corresponding DNA binding boxes, has led to the breaking of the TALE proteins DNA binding code. The number of DBD repeats may range from 1.5 to 30. The repeat variable di-residue (RVD), at positions 12 and 13 of each repeat, dictates the specificity of the binding to one nucleotide in the DNA target, with the one at position 13 actually binding to the nucleotide. In addition, the first RVD must recognize a “T” nucleotide located right before the binding site for binding to occur. FIG. 4A shows a list of known RVDs 402 that bind to each of the possible nucleotides 404. FIG. 4B shows the list of known RVDs 406 together with their known binding specificities 408 and known efficiencies to promote TALE activity 410. For example, the “HD” RVD binds to a “C” nucleotide with a relatively strong efficiency or binding strength, while the “NN” binds more to a “G” than an “A”, binding to the former with a relatively strong binding strength and the latter with an intermediate binding strength.

For a given genomic, the design of a TALEN or TALEN pair may depend on a variety of factors. In addition to the requirements discussed above, such as including an N-terminal secretion signal, a nuclear localization signal, an acidic activation domain at the C-terminal, and having appropriate RVDs in each repeat that bind to a region of the given genomic, the system disclosed in the present application also takes at least some of the following factors into consideration. (1) TALE length or the number of repeats. While the number of repeats in a TALE may generally vary within a large range, it has been shown experimentally that about 6 to about 40, about 10 to about 30, about 14 to about 21, or about 15 to about 20 repeats work well in terms of sequence specificity and ease of experimental design. (2) Spacer length. Generally, a DCD needs sufficient room to bind to a DNA region and perform cleavage. In addition, when two DBDs are closer, the corresponding two DCDs are more likely to be properly situate themselves and their dimerization is thus more likely to occur. It has been shown experimentally that 14-16 residues corresponding to 14-16 base pairs in the spacer region work well. (3) Last RVD. It has been experimentally shown that the last nucleotide to which a DBD binds is typically a “T”, and it can be helpful to use the “NG”, whose binding efficiency is generally known, as the last RVD in the DBD. (4) GC content. As binding of a RVD with a “G” or “C” nucleotide is generally with a higher efficiency and specificity than binding with an “A” or a “T”, it is preferable to include in a certain proportion of the repeats, such as 30%-70%, RVDs that tend to bind with a “G” or a “C”, include, for example, “HD” and “NH”. (5) First RVDs. As demonstrated in experiments, it is desirable to have some of the initial RVDs, such as two out of the first three, to bind with a “G” or a “C” with a strong specificity and efficiency. (6) Uniqueness. It is possible that a TALEN binds to multiple locations in the given genome. It may be desirable to achieve higher specificity with one or both of a pair of TALENs binding to a small number of locations and minimize on “off-target” interaction. (7) Mononucleotide repeats. Mononucleotide repeats tend to occur heavily in repetitive DNA and thus are not ideal for achieving specificity. In addition, mononucleotide independence within TALE target sites was experimentally observed. Furthermore, mononucleotide repeats may slightly distort DNA and thus affect binding. It therefore may be helpful to disregard TALENs that bind to consecutive “G” or “C” nucleotides and especially consecutive “A” or “T” nucleotides, the latter significantly affecting the overall binding strength.

### Computational System and Methods

This application presents a system and related methods that determine candidate residue sequences for transcription activator-like effector nucleases for genome cleavage tasks. In some embodiments, the system comprises one or more servers connected with one or more memories, which can be implemented by a cloud-computing platform, a server farm, a parallel-computing device, and so on having sufficient computing and storage power to efficiently process a large number of DNA and protein sequences and other types of data. The system can include input and output devices, and it can also include client devices for interacting with the servers across communication networks, which can be implemented by a desktop computer, a laptop computer, a tablet, a cellphone, a wearable device, and other smart user electronic devices. Examples of the communication networks include the Internet, a cellular network, a short-range Bluetooth network, etc.

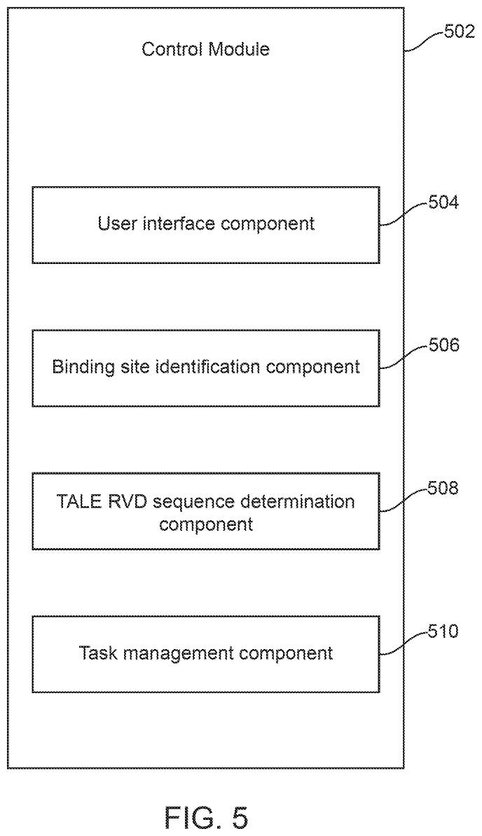

FIG. 5 illustrates example computer components that can be used for implementing the system disclosed in this application. In some embodiments, the system comprises a control module 502 that controls various components, including a user interface component 504, a binding site identification component 506, a TALE RVD sequence determination component 508, and a task management component 510. These modules can be directly implemented in hardware or as one or more software programs. The user interface component 504 handles user input and output, through a graphical user interface (GUI), an application programming interface (API), or other means. A networking component that handles network communication can be incorporated into this component or set up as a separate component. The binding site identification component 506 handles identification of binding sites within a DNA region for TALE DBDs. The TALE RVD sequence determination component 508 handles determination of residue sequences for the TALE DVDs that may bind to the identified binding sites. The task management component 510 interacts with the other components based on the given genome engineering task, which can be a single-hit task, an excision task, a strafe task, an imaging task, and so on. Various details of the communication among the components are presented below.

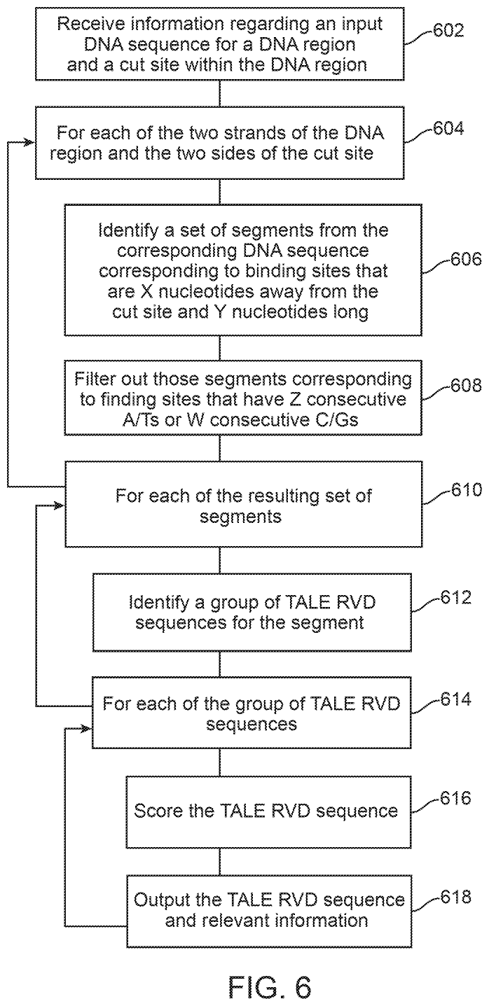

FIG. 6 illustrates an example process performed by the system of generating a pair of TALE RVD sequences for TALEN cleavage. In some embodiments, the system can build an index for a reference genome or a given genome in advance that indicates, for each of the four nucleotides, the locations within the genome where the nucleotide occurs. In step 602, the system receives from a user or a client system over a network information regarding an input DNA sequence for a DNA region that is largely identical to a region within the reference genome or is from the given genome and information regarding a cut site within the input DNA sequence. The information can be submitted through a GUI or an API provided by the system. The input DNA sequence may be provided in its entirety or by specifying a start position and an end position within the reference or given genome. Since a pair of TALENs that performs a cleavage bind to different stands, the system also produces the complimentary DNA sequence. The cut site may also be expressed as a position within the reference or given genome or the input DNA sequence.

In some embodiments, in steps 604-618, the system examines the DNA bases and determines candidate TALE RVD sequences for each of the two (input and complementary) DNA sequences corresponding to TALEN binding sites on each of the two sides of the cut site. In step 606, the system first identifies a set of fragments from the DNA sequence corresponding to TALEN binding sites that are X nucleotides away from the cut site and Y nucleotides long. Since a “T” nucleotide must be present right before a binding site, the system can start with only those fragments that are preceded by a “T” using the pre-built index. X is related to the length of the spacer region between two TALEN binding sites. For example, X can be 5-10 (leading to a spacer region of 10 to 20 nucleotides) or whatever range that is biologically feasible. Y is related to the DBD length of a TALEN or more specifically the length of a TALEN RVD sequence. For example, Y can be 6-40 or whatever range is biologically feasible. In step 608, the system then filters out those fragments corresponding to TALEN binding sites that have Z consecutive “A” or “T” nucleotides or W consecutive “G” or “C” nucleotides. Z and W are related to the length of mononucleotide repeats in a TALEN binding site. Z and W may have the same or different values, such as 5 and 7, respectively. Upon completing steps 610-618, the system returns to step 606.

In steps 610-618, the system determines corresponding candidate TALE RVD sequences for each of the remaining fragments. In step 612, the system identifies a group of candidate TALE RVD sequences corresponding to DBDs that may bind to the binding site represented by the fragment according to FIG. 4A. The binding specificities shown in FIG. 4 can be converted into numerical or categorical values. The system can set a threshold on binding specificities and consider a TALE RVD only if the binding occurs with a binding specificity above the threshold. For example, binding specificity of a TALE RVD may be measured by how many times a particular TALE RVD maps to or binds to a genome (optionally, allowing for a small number of base mismatches, such as 1 or 2). For example, the threshold for binding specificity may be set such that a TALE RVD maps to or binds to the genome at no more than 1, 2, 3, 4, 5, 6, 7, 8, 9, 10, or more than 10 unique locations). The system may consider only a certain number of TALE RVDs, such as the first N RVDs with the highest binding affinity (strongest binding) or the highest binding specificity, where N is an integer such as 1, 2, 3, 4, 5, 6, 7, 8, 9, 10, 11, 12, 13, 14, 15, 16, 17, 18, 19, 20, or more than 20). Upon completing steps 614-518, the system returns step 612.

In steps 614-618, the system generates a score for each of the candidate TALE RVD sequences. In step 616, the system assigns a score to the candidate TALE RVD sequence using a scoring function, as discussed below. In step 618, the system outputs the TALE RVD sequence and relevant information. The output can be transmitted back to the client device (e.g., over the network) and/or presented through the GUI or the API. The output can include the score and basic information regarding the binding site, such as a position within the input sequence or the given or reference genome, an identification of the strand (input or complementary DNA sequence), etc. The output can also include details or summary statistics related to the different factors discussed above, such as the number of repeats, the spacer length, the proportion of RVDs throughout or in the first three repeats that bind to a “G” or a “C”, the number of binding sites in the reference or given genome, and so on.

The set of candidate TALE RVD sequences may be ordered or ranked according to their assigned score which is generated using the scoring function. Alternatively or in combination, the set of candidate TALE RVD sequences may be filtered (e.g., a subset of candidate TALE RVD sequences may be removed from the set) according to their assigned score. For example, candidate TALE RVD sequences with scores below a threshold value may be removed. Alternatively or in combination, the set of candidate TALE RVD sequences may be classified according to their assigned score. For example, candidate TALE RVD sequences with scores below a threshold value may be classified as “weak” and candidate TALE RVD sequences with scores above a threshold value may be classified as “strong.” As another example, candidate TALE RVD sequences with scores below a first threshold value may be classified as “weak,” candidate TALE RVD sequences with scores between the first threshold value and a second threshold value may be classified as “intermediate,” and candidate TALE RVD sequences above the second threshold value may be classified as “strong.” Candidate TALE RVD sequences may be further processed based on their ordering or ranking, and/or based on their classification as a “weak,” “intermediate,” or “strong” candidate. For example, “strong” candidate TALE RVD sequences may be used to synthesize TALENs, using methods such as those described herein. The system may advantageously identify low-scoring or “weak” candidate TALE RVD sequences for exclusion from synthesis and testing, thereby providing significant gains in throughput and/or reduction in development costs.

In some embodiments, the scoring function assigns a total score to a TALE RVD sequence based on one or more of the following conditions related to the factors discussed above. The scoring function may generate a score based on any set of 1, 2, 3, 4, 5, 6, or 7 of the following conditions or factors, by assigning a higher score when the conditions satisfy certain criteria. (1) TALE length or number of repeats. A sequence may receive a higher score when its length is between about 14 and about 21, or between about 15 and about 20, and a lower score otherwise. (2) Spacer length. A sequence may receive a higher score when the distance from the corresponding binding site to the cut site (cleavage position) is about 14-16 base pairs, and a lower score otherwise. (3) Last RVD. A sequence may receive a higher score when its last RVD is “NG”, an intermediate score when its last RVD is not “NG” but corresponds to a “T” according to FIG. 4A, or a low score otherwise. (4) GC content of RVDs. A sequence may receive a higher score when it has a larger number of RVDs (e.g., 1, 2, 3, 4, 5, 6, 7, 8, 9, 10, or more than 10) that correspond to a “G” or a “C”, and a lower score otherwise. (5) First RVDs. A sequence may receive a higher score when a larger number of the first N (a positive number, such as 1, 2, 3, 4, 5, 6, 7, 8, 9, 10, or more than 10) RVDs correspond to a “G” or a “C”, and a lower score otherwise. (6) Uniqueness of binding sites in a reference or given genome. A sequence may receive a score that is inversely proportional to the number of corresponding binding sites in the reference or given genome. (7) Number of mononucleotide repeats. When this condition is not used as an initial filter, the scoring function can assign a score to a sequence that is inversely proportional to the length of any series of consecutive RVDs included by the sequence that correspond to a “G” or a “C” or that correspond to a “T” or an “A”. Similarly, any of the other conditions or factors may be used as an initial filter rather than incorporated into the scoring function.

In some embodiments, when an individual score is related to binding, the scoring function further differentiates the score based on the binding specificity or efficiency, as shown in FIG. 4B. For example, since an “HD” binds to only a “C” with a strong efficiency, while an “NS” binds to a “C” as well as other nucleotides with an intermediate efficiency, an “HD” may warrant a higher score than an “NS” with respect to the fourth or fifth factor discussed above. The binding specificities or efficiencies can also be used to adjust the identification of the fragments within the (input or complementary) DNA sequence that correspond to binding sites. For example, since an “NS” may be more inclined to bind to a “G” or an “A” than a “C” or a “T”, a binding site where an “NS” may bind to a “C” or a “T” may be ignored. In some cases, the scoring function associates much higher scores with RVDs that specifically bind only single nucleotides and are tight binders for those nucleotides, such as “HD”, “NH”, “NI”, and “NH”.

In some embodiments, the scoring function may generate each individual score by imposing a probability distribution, such as a normal distribution, on the range of possible values so that the highest probability becomes the score of the most favorable value. The scoring function may assign a weight to each individual score to prioritize the factors as desired by an administrator, an end user, and so on. Each of the weights may be zero or non-zero. A weight of zero may be applied to factors that are not used in the weighted score (or were used elsewhere such as for filtering TALE RVD sequences before or after scoring), and a non-zero weight may be applied to factors that are used in the weighted score. In some cases, the scoring function focuses on (4) the GC content, (5), the first RVDs, and/or (7) the mononucleotide repeats. For example, the scoring function S may be given by:

S=0.33(a)+0.33(b)+0.33(c),

Here, a may correspond to the strength of the start defined by: a=0.33(n1)+0.33(n2)+0.33(n3)+0.33((n4+n5)/2), where n1, n2, n3, n4, and n5 (corresponding to the first 5 RVDs) have values of 1 when the RVDs are strong binders and 0 when they are weak binders. While a can be >1, it is rounded down to 1 in such cases. In addition, b may correspond to the GC content in terms of the percentage of nucleotides being G or C in the binding site. Moreover, c may be set to values of 1 or 0 depending on whether or not there are any mononucleotide runs (As and Ts>5 and Gs and Cs>8) in the binding site. In this example, S results in a score between 0 and 1. The scoring function can be refined by also focusing on (1) the TALE length or (2) the spacer length. For example, S can produce a score of 0 unless the TALEN has between 15-21 RVDs and a corresponding spacer length between 14-16 base pairs. As another example, S can produce a score of 0 unless the TALEN has a unique binding site in the genome.

In some embodiments, the values for the TALENs in a pair are averaged to give a score for a pair of TALENs. It can be appreciated by someone of ordinary skill in the art that this is merely an example, and different weights in the formulas, different numbers of initial RVDs, different mononucleotide run lengths, different score ranges, and so on can be used.

By virtue of the features described above, the system may allow a user to select a TALE RVD sequence for one strand of a DNA region on one side (e.g., a first side) of the cut site, a TALE RVD sequence for the other strand on the other side of the cut site, and generate a pair of TALENs by generating a TALE based on each of the selected TALE RVD sequences and connecting each TALE with an appropriate signal and other additional elements so that the two TALENs may combine to cut at the cut site.

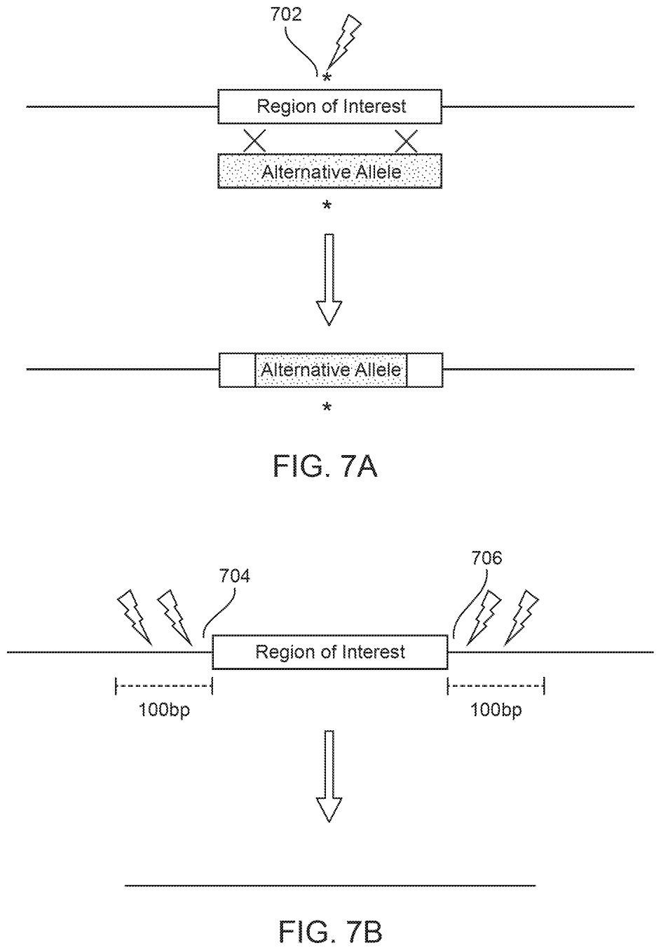

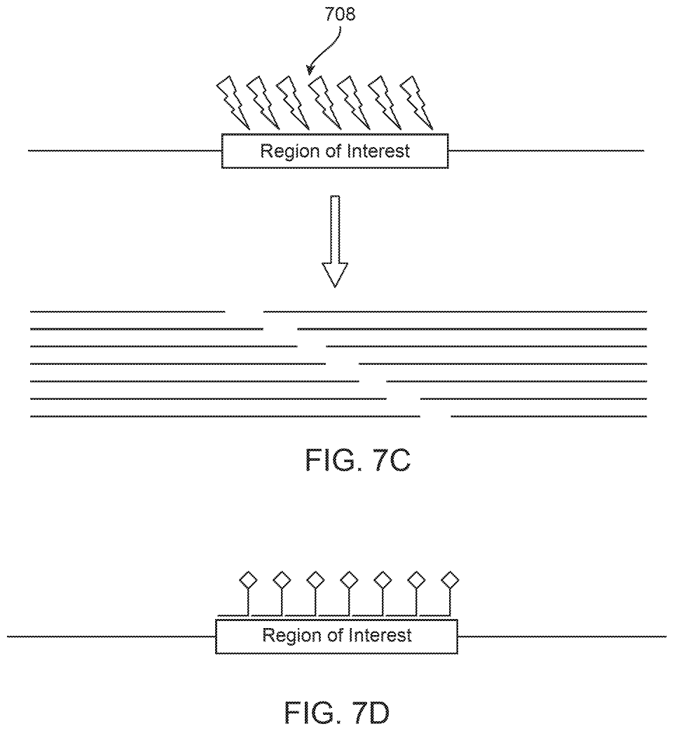

The ability of TALENs to bind to specific DNA regions and to perform cleavage at specific positions within DNA regions can be applied to a variety of genome engineering tasks. FIG. 7A illustrates an example application of TALEN cleavage for a single-hit task. A process such as one illustrated in FIG. 6 can be used to design a pair of TALENs that may cut at a specific site 702. One or more base pairs can then be inserted at the cut site for genome alteration, repair, or other purposes. FIG. 7B illustrates an example application of TALEN cleavage for a flank/excision task. Similarly, the same process can be applied repeatedly to design two pairs of TALENs that respectively cut at two sites 704 and 706 within a genome to excise the region between the two sites also for various engineering purposes. FIG. 7C illustrates an example application of TALEN cleavage for a strafe task. Furthermore, the same process can be applied repeatedly to design a series of pairs of TALENs that cut at successively positions 708 within a DNA region to evaluate functional implications of deleting individual base pairs within the region. FIG. 7D illustrates an example application of TALEN cleavage for an imaging task. In addition, the same process can be modified to design, instead of a pair of TALENs that bind to separate stands, a series of TALENs that bind to successive regions (displaced by one nucleotide each time) of the same strand. Each TALE is fused with a florescent component, such as a green florescent protein (GFP), instead of a nuclease for special imaging purposes.

### Computer Systematization

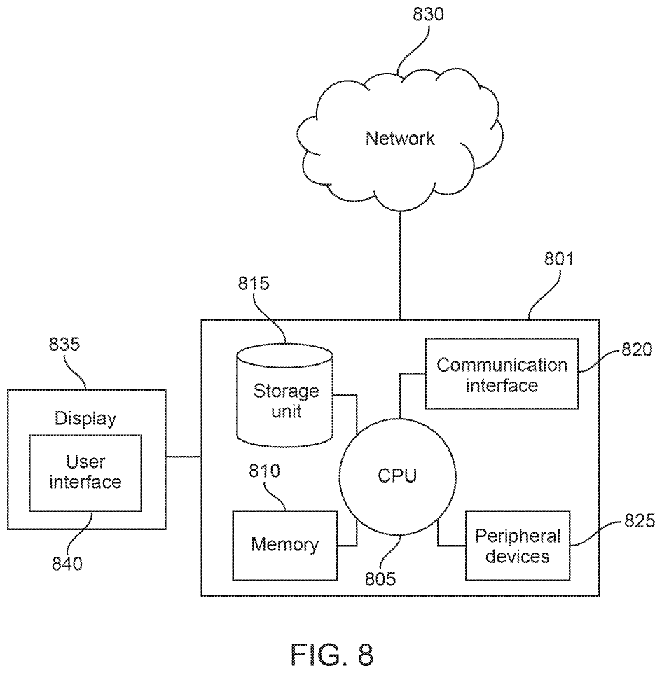

FIG. 8 shows a computer system 801 that can be configured to implement any computing system disclosed in the present application. The computer system 801 can comprise a mobile phone, a tablet, a wearable device, a laptop computer, a desktop computer, a central server, etc.

The computer system 801 includes a central processing unit (“CPU”, also “processor” and “computer processor” herein) 805, which can be a single core or multi core processor, or a plurality of processors for parallel processing. The computer system 801 also includes memory or memory location 810 (e.g., random-access memory, read-only memory, flash memory), electronic storage unit 815 (e.g., hard disk), communication interface 820 (e.g., network adapter) for communicating with one or more other systems, and peripheral devices 825, such as cache, other memory, data storage and/or electronic display adapters. The memory 810, storage unit 815, interface 820 and peripheral devices 825 are in communication with the CPU 805 through a communication bus (solid lines), such as a motherboard. The storage unit 815 can be a data storage unit (or data repository) for storing data. The computer system 801 can be operatively coupled to a computer network (“network”) 830 with the aid of the communication interface 820. The network 830 can be the Internet, an internet and/or extranet, or an intranet and/or extranet that is in communication with the Internet. The network 830 in some cases is a telecommunication and/or data network. The network 830 can include one or more computer servers, which can enable distributed computing, such as cloud computing. The network 830, in some cases with the aid of the computer system 801, can implement a peer-to-peer network, which may enable devices coupled to the computer system 801 to behave as a client or a server.

The CPU 805 can execute a sequence of machine-readable instructions, which can be embodied in a program or software. The instructions may be stored in a memory location, such as the memory 810. The instructions can be directed to the CPU 805, which can subsequently program or otherwise configure the CPU 805 to implement methods of the present disclosure. Examples of operations performed by the CPU 805 can include fetch, decode, execute, and writeback.

The CPU 805 can be part of a circuit, such as an integrated circuit. One or more other components of the system 801 can be included in the circuit. In some cases, the circuit is an application specific integrated circuit (ASIC).

The storage unit 815 can store files, such as drivers, libraries and saved programs. The storage unit 815 can store user data, e.g., user preferences and user programs. The computer system 801 in some cases can include one or more additional data storage units that are external to the computer system 801, such as located on a remote server that is in communication with the computer system 801 through an intranet or the Internet.

The computer system 801 can communicate with one or more remote computer systems through the network 830. For instance, the computer system 801 can communicate with a remote computer system of a user. Examples of remote computer systems include personal computers, slate or tablet PC's, smart phones, personal digital assistants, and so on. The user can access the computer system 801 via the network 830.

Methods as described herein can be implemented by way of machine (e.g., computer processor) executable code stored on an electronic storage location of the computer system 801, such as, for example, on the memory 810 or electronic storage unit 815. The machine executable or machine-readable code can be provided in the form of software. During use, the code can be executed by the processor 805. In some cases, the code can be retrieved from the storage unit 815 and stored on the memory 810 for ready access by the processor 805. In some situations, the electronic storage unit 815 can be precluded, and machine-executable instructions are stored on memory 810.

The code can be pre-compiled and configured for use with a machine having a processor adapted to execute the code, or can be compiled during runtime. The code can be supplied in a programming language that can be selected to enable the code to execute in a pre-compiled or as-compiled fashion.

Aspects of the systems and methods provided herein, such as the computer system 801, can be embodied in programming. Various aspects of the technology may be thought of as “products” or “articles of manufacture” typically in the form of machine (or processor) executable code and/or associated data that is carried on or embodied in a type of machine-readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links, or the like, also may be considered as media bearing the software. As used herein, unless restricted to non-transitory, tangible “storage” media, terms such as computer or machine “readable medium” refer to any medium that participates in providing instructions to a processor for execution.

Hence, a machine-readable medium, such as computer-executable code, may take many forms, including but not limited to, a tangible storage medium, a carrier wave medium, or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 801 can include or be in communication with an electronic display 835 that comprises a user interface 840 for providing, for example, a management interface. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 805.

### Certain Terminologies

Unless defined otherwise, all technical and scientific terms used herein have the same meaning as is commonly understood by one of skill in the art to which the claimed subject matter belongs. It is to be understood that the detailed description are exemplary and explanatory only and are not restrictive of any subject matter claimed. In this application, the use of the singular includes the plural unless specifically stated otherwise. It must be noted that, as used in the specification, the singular forms “a,” “an” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

Although various features of the invention may be described in the context of a single embodiment, the features may also be provided separately or in any suitable combination. Conversely, although the invention may be described herein in the context of separate embodiments for clarity, the invention may also be implemented in a single embodiment.

Reference in the specification to “some embodiments”, “an embodiment”, “one embodiment” or “other embodiments” means that a particular feature, structure, or characteristic described in connection with the embodiments is included in at least some embodiments, but not necessarily all embodiments, of the inventions.

As used herein, ranges and amounts can be expressed as “about” a particular value or range. About also includes the exact amount. Hence “about 5 μL” means “about 5 μL” and also “5 μL.” Generally, the term “about” includes an amount that may be expected to be within experimental error.

The section headings used herein are for organizational purposes only and are not to be construed as limiting the subject matter described.

### EXAMPLES

These examples are provided for illustrative purposes only and not to limit the scope of the claims provided herein.

### Example 1

### Exemplary TALEN Assembly Methodology

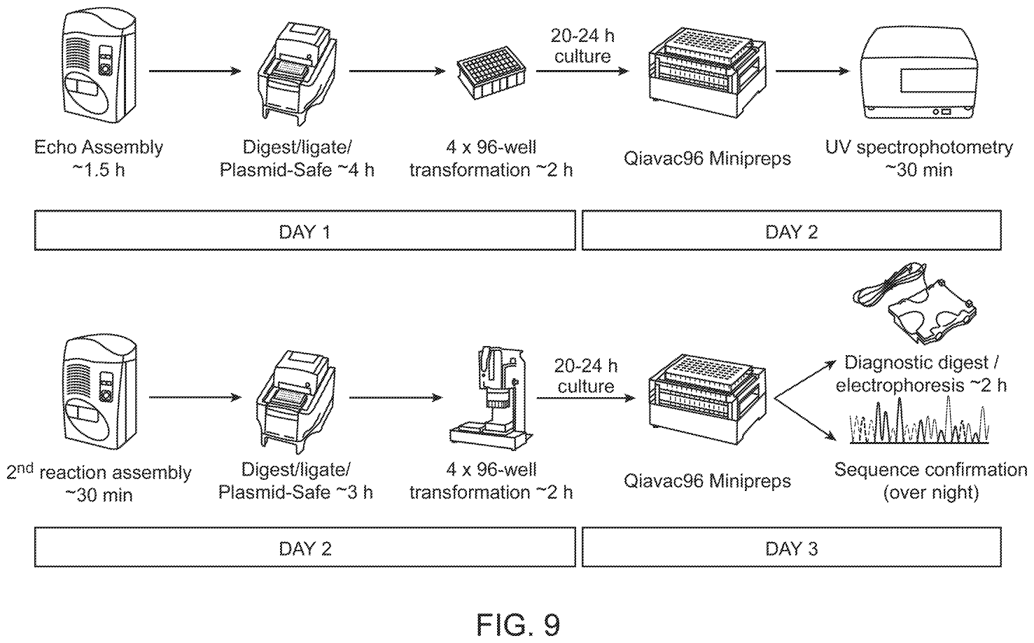

A high-throughput assembly pipeline can employ an acoustic delivery ejection technology (e.g., utilizing a high-throughput acoustic liquid handler instrument, such as a Labcyte Echo 550) to assemble proteins of interest en masse. The high-throughput methodology can further enable the proteins of interest to be generated in about 3 days. For example, the high-throughput methodology can enable one to rapidly and efficiently assemble about 100 or more TALEN dimers per week, as compared to a throughput of a few (about 2 to 4) TALEN dimers per week using previous lower-throughput approaches. The assembly can involves two steps: assembly of an array of intermediary repeat units each comprising about 1-6 repeats and joining of the intermediary arrays into a backbone to generate the final polypeptide of interest. The following example provides a protocol for generation of TALENs. FIG. 9 illustrates the schematics of the assembly protocol.

Day 1 Assembly:

The assembly protocol was generated using EchoTools. The reaction mixture was assembled based on Table 3 on a 384-well plate.

TABLE 3
Digest/Ligation - 2 μL final volume

| | Vol. (μL) |
|---|---|
| RVD #1 (75 ng/μL) | 0.2 |
| RVD #2 (75 ng/μL) | 0.2 |
| RVD #3 (75 ng/μL) | 0.2 |
| RVD #4 (75 ng/μL) | 0.2 |
| RVD #5 (75 ng/μL) | 0.2 |
| RVD #6 (75 ng/μL) | 0.2 |
| pFUS (75 ng/μL) | 0.2 |
| BsaI | 0.1 |
| T4 DNA ligase | 0.1 |
| 10× T4 DNA ligase | 0.2 |
| buffer | |
| 10× BSA | 0.2 |
| TOTAL | 2 μL |

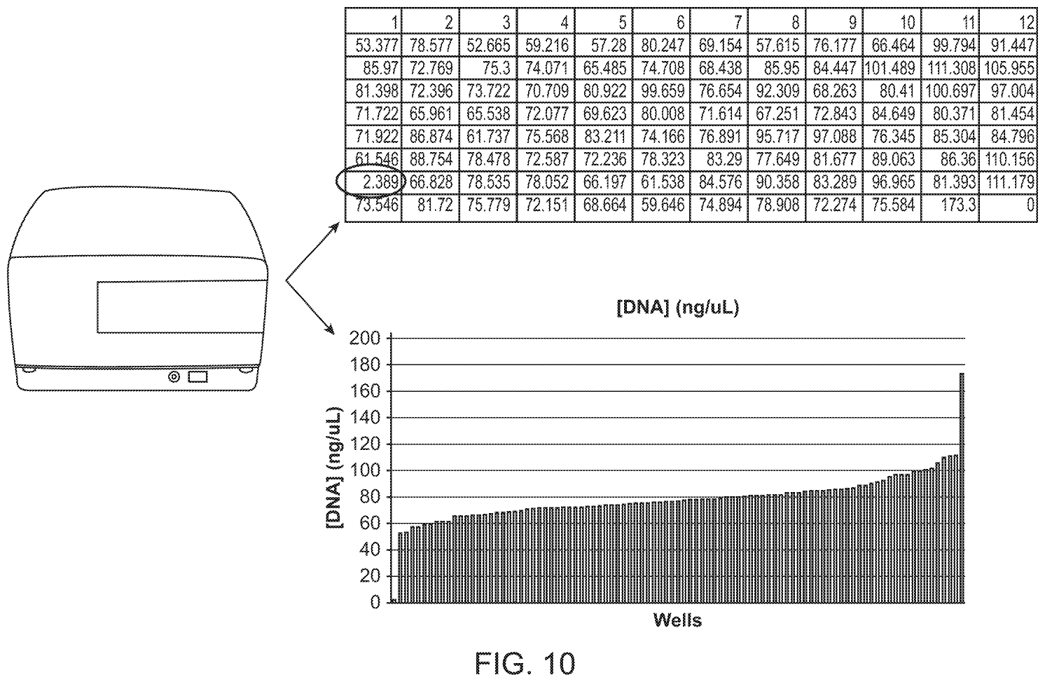

After assembly, the 384-well plate was incubated in a thermocycler for about 10 cycles of about 5 min at 37° C. for digestion and about 10 min at 16° C. for ligation. After each cycle, the reaction mixture was further heated to about 50° C. for about 5 min and then to about 80° C. for about 5 min to reduce background. After the digestion and ligation step, about 1 μL of 20 mM ATP, 1 μL of Plasmid Safe DNase (10U, Epicentre) and 1 μL of BsaI-HF were added into the reaction mixture, and further incubated for at least 1 hour at 37° C. Treatment with Plasmid Safe DNase and BsaI-HF can enable removal of empty vectors and non-ligated plasmids. The treated reaction mixture was then used to transform Clontech Stellar cells. The transformed Clontech Stellar cells were incubated in a 96-well format with LB at 700 rpm and 37° C. for up to 20-24 hours. Miniprep was performed on the 96-well culture and DNA concentrations were measured using a UV spectrophotometry (FIG. 10).

Day 2 Assembly:

Day 2 reaction mixture was assembled according to Table 4.

TABLE 4
Digest/Ligation #2

| | Vol |
|---|---|
| | (μL) |
| pFUS-A2A (~150 ng/μL) | 0.2 |
| pFUS-A2B (~150 ng/μL) | 0.2 |
| pFUS-B (~150 ng/μL) | 0.2 |
| (Additional pFUS-A3A/B or | 0.2 |
| MQW) | |
| pVax_LR-NG63aa (~75 ng/μL) | 0.2 |
| BsmBI | 0.1 |
| T4 DNA ligase | 0.1 |
| 10× T4 DNA ligase buffer | 0.2 |
| MQW | 0.6 |
| TOTAL | 2 |

The Day 2 reaction mixtures were assembled on a 384-well plate and was incubated in a thermocycler for about 10 cycles accordingly to the Day 1 protocol. The pVax vector can contain a pre-assembled polynucleotide region that encodes a C-terminal half-repeat and a polynucleotide region that encodes FokI. After the digestion and ligation step, about 1 μL of 20 mM ATP, 1 μL of Plasmid Safe DNase (10U, Epicentre), and 1 μL of BsaI-HF were added into the reaction mixture, and further incubated for at least 1 hour at 37° C. The treated reaction mixture was then used to transform Clontech Stellar cells. The transformed Clontech Stellar cells were incubated in a 96-well format with LB at 700 rpm and 37° C. for up to 20-24 hours.

Day 3:

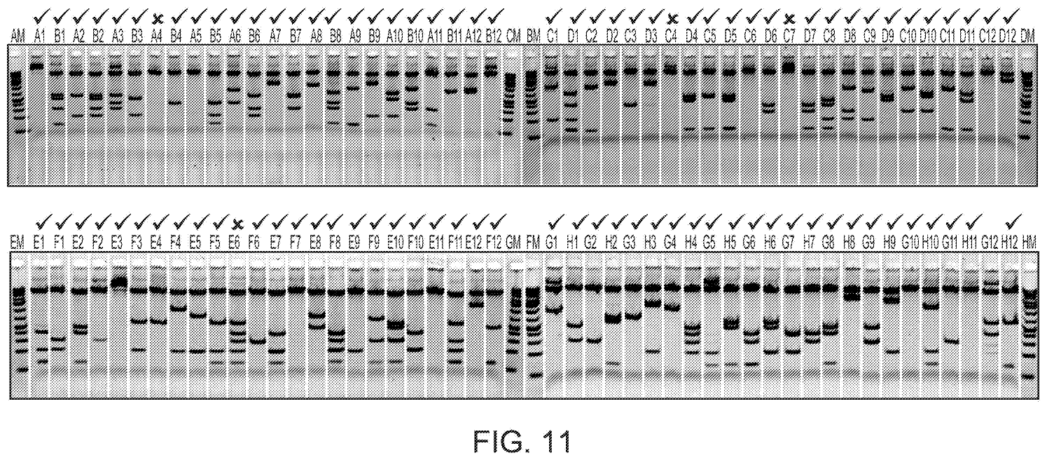

Miniprep was performed on the 96-well culture on Day 3. The DNA elutes were analyzed either by electrophoresis (FIG. 11) or by sequence confirmation.

### Example 2

### Exemplary Nucleic Acid Assembly

A high-throughput assembly pipeline employing an acoustic delivery ejection technology (e.g., utilizing a high-throughput acoustic liquid handler instrument, such as a Labcyte Echo 550) can be used to assemble nucleic acids of interest en masse. The assembly can involve two steps: assembly of an array of intermediary nucleic acid fragments and joining of the intermediary nucleic acid fragments into a backbone to generate the array of nucleic acids of interest.

The assembly protocol can be generated using EchoTools. A first set of reaction mixtures is assembled based on Table 5 on a 384-well plate.

TABLE 5
Digest/Ligation - 2 μL final volume

| | Vol. (μL) |
|---|---|
| Nucleic acid fragment #1 | 0.2 |
| Nucleic acid fragment #2 | 0.2 |
| Nucleic acid fragment #3 | 0.2 |
| pFUS (75 ng/μL) | 0.2 |
| BsaI | 0.1 |
| T4 DNA ligase | 0.1 |
| 10× T4 DNA ligase buffer | 0.2 |
| 10× BSA | 0.2 |
| MQW | 0.6 |
| TOTAL | 2 μL |

After assembly, the 384-well plate is incubated in a thermocycler for about 10 cycles of about 5 min at 37° C. for digestion and about 10 min at 16° C. for ligation. After each cycle, the reaction mixture is further heated to about 50° C. for about 5 min and then to about 80° C. for about 5 min to reduce background. After the digestion and ligation step, about 1 μL of 20 mM ATP, 1 μL, of Plasmid Safe DNase (10U, Epicentre), and 1 μL of BsaI-HF are added into the reaction mixture, and further incubated for at least 1 hour at 37° C. Treatment with Plasmid Safe DNase and BsaI-HF can enable removal of empty vectors and non-ligated plasmids. The treated reaction mixture is then used to transform Clontech Stellar cells. The transformed Clontech Stellar cells are incubated in a 96-well format with LB at 700 rpm and 37° C. for up to 20-24 hours. Miniprep is performed on the 96-well culture and nucleic acid concentrations are measured using a UV spectrophotometry.

After miniprep and measurement of nucleic acid concentration, a second set of reaction mixtures is assembled according to Table 6.

TABLE 6
Digest/Ligation #2

| | Vol |
|---|---|
| | (μL) |
| pFUS-intermediate fragment 1 | 0.2 |
| pFUS-intermediate fragment 2 | 0.2 |
| pFUS-intermediate fragment 3 | 0.2 |
| pVax (~75 ng/μL) | 0.2 |
| BsmBI | 0.1 |
| T4 DNA ligase | 0.1 |
| 10× T4 DNA ligase buffer | 0.2 |
| MQW | 0.8 |
| TOTAL | 2 |

The second set of reaction mixtures is assembled on a 384-well plate and is incubated in a thermocycler for about 10 cycles accordingly to the protocol above. After the digestion and ligation step, about 1 μL of 20 mM ATP, 1 μL of Plasmid Safe DNase (10U, Epicentre), and 1 μL of BsaI-HF are added into the reaction mixture, and further the reaction mixture is further incubated for at least 1 hour at 37° C. The treated reaction mixture is then used to transform Clontech Stellar cells. The transformed Clontech Stellar cells are incubated in a 96-well format with LB at 700 rpm and 37° C. for up to 20-24 hours.

Miniprep is performed on the 96-well culture. The nucleic acid elutes are analyzed either by electrophoresis or by sequence confirmation.

### Example 3

Table 7 illustrates an exemplary FokI sequence that can be used herein with a method or system described herein.

TABLE 7

| | SEQ |
|---|---|
| FokI | ID NO: |
| MFLSMVSKIRTFGWVQNPGKFENLKRVVQVFDRNSKVHNEVK | 1 |
| NIKIPTLVKESKIQKELVAIMNQHDLIYTYKELVGTGTSIRS | |
| EAPCDAIIQATIADQGNKKGYIDNWSSDGFLRWAHALGFIEY | |
| INKSDSFVITDVGLAYSKSADGSAIEKEILIEAISSYPPAIR | |
| ILTLLEDGQHLTKFDLGKNLGFSGESGFTSLPEGILLDTLAN | |
| AMPKDKGEIRNNWEGSSDKYARMIGGWLDKLGLVKQGKKEFI | |
| IPTLGKPDNKEFISHAFKITGEGLKVLRRAKGSTKFTRVPKR | |
| VYWEMLATNLTDKEYVRTRRALILEILIKAGSLKIEQIQDNL | |
| KKLGFDEVIETIENDIKGLINTGIFTEIKGRFYQLKDHILQF | |
| VIPNRGVTKQLVKSELEEKKSELRHKLKYVPHEYIELIEIAR | |
| NSTQDRILEMKVMEFFMKVYGYRGKHLGGSRKPDGAIYTVGS | |
| PIDYGVIVDTKAYSGGYNLPIGQADEMQRYVEENQTRNKHIN | |
| PNEWWKVYPSSVTEFKFLFVSGHFKGNYKAQLTRLNHITNCN | |
| TLTLEEVRRKFNNGEINFGAVLSVEELLIGGEMIKAG | |

The examples and embodiments described herein are for illustrative purposes only and various modifications or changes suggested to persons skilled in the art are to be included within the spirit and purview of this application and scope of the appended claims.

What is claimed is:

1. A computer-implemented method of determining protein sequences for genome engineering, comprising:
receiving input information regarding an input DNA sequence for a DNA region in a given genome containing binding sites for proteins and a cleavage position for the proteins within the DNA region;
identifying a plurality of fragments of the input DNA sequence respectively corresponding to a plurality of the binding sites to a first side of the cleavage position;
determining a plurality of protein di-residue sequences for a plurality of the proteins to bind to the plurality of binding sites based on specificity information related to binding of protein di-residues to DNA bases;
assigning a score to each of the plurality of protein di-residue sequences with a scoring function that generates a score based on at least one of the following conditions of the protein di-residue sequence:
(a) TALE length or number of repeats;
(b) spacer length;
(c) last repeat variable dinucleotide (RVD);
(d) GC content of RVDs;
(e) first RVDs;
(f) uniqueness of binding sites in the given genome; or
(g) number of mononucleotide repeats; and
generating output information regarding the plurality of protein di-residue sequences, including the assigned scores.

2. The computer-implemented method of claim 1, wherein the scoring function generates the score based on at least two of the conditions (a) through (g).

3. The computer-implemented method of any of claims 1-2, wherein the scoring function generates the score based on at least three of the conditions (a) through (g).

4. The computer-implemented method of any of claims 1-3, wherein the scoring function generates the score based on at least four of the conditions (a) through (g).

5. The computer-implemented method of any of claims 1-4, wherein the scoring function generates the score based on at least five of the conditions (a) through (g).

6. The computer-implemented method of any of claims 1-5, wherein the scoring function generates the score based on at least six of the conditions (a) through (g).

7. The computer-implemented method of any of claims 1-6, wherein the scoring function generates the score based on all of the conditions (a) through (g).

8. The computer-implemented method of any of claims 1-7, wherein the scoring function generates a higher score when the TALE length or number of repeats of the protein di-residue sequence is between 14 and 21.

9. The computer-implemented method of any of claims 1-7, wherein the spacer length of the protein di-residue sequence comprises a distance from a corresponding binding site of the protein di-residue sequence to the cleavage position of the protein di-residue sequence.

10. The computer-implemented method of claim 9, wherein the scoring function generates a higher score when the spacer length of the protein di-residue sequence is 14 to 16 base pairs.

11. The computer-implemented method of any of claims 1-7, wherein the scoring function generates a higher score when the last repeat variable dinucleotide (RVD) of the protein di-residue sequence is “NG.”

12. The computer-implemented method of any of claims 1-7, wherein the scoring function generates a higher score when the last repeat variable dinucleotide (RVD) of the protein di-residue sequence is not “NG” but corresponds to a “T” according to FIG. 4A.

13. The computer-implemented method of any of claims 1-7, wherein the scoring function generates a higher score when the GC content of RVDs of the protein di-residue sequence comprises a number of RVDs of the protein di-residue sequence that correspond to a “G” or a “C.”

14. The computer-implemented method of claim 13, wherein the scoring function generates a higher score when the GC content of RVDs of the protein di-residue sequence is 1 to 10 RVDs.

15. The computer-implemented method of any of claims 1-7, wherein each of the first N RVDs of the protein di-residue sequence corresponds to a “G” or a “C.”

16. The computer-implemented method of claim 15, where the scoring function generates a higher score when N is 1 to 10.

17. The computer-implemented method of any of claims 1-7, wherein the uniqueness of binding sites in the given genome of the protein di-residue sequence comprises a number of corresponding binding sites in the given genome of the protein di-residue sequence.

18. The computer-implemented method of claim 17, wherein the scoring function is inversely proportional to the uniqueness of binding sites in the given genome of the protein di-residue sequence.

19. The computer-implemented method of any of claims 1-7, wherein the number of mononucleotide repeats comprises a length of any series of consecutive RVDs in the protein di-residue sequence that correspond to a “G” or a “C” or that correspond to a “T” or an “A.”

20. The computer-implemented method of claim 19, wherein the scoring function is inversely proportional to the number of mononucleotide repeats of the protein di-residue sequence.

21. The computer-implemented method of any of claims 1-20, wherein at least one of the conditions (a) through (g) is used as an initial filter applied to the plurality of protein di-residue sequences.

22. The computer-implemented method of any of claims 1-21, wherein the input information includes a start position and an end position of the DNA region within the given genome.

23. The computer-implemented method of any of claims 1-21, wherein each of the plurality of binding sites satisfies a length requirement and a location requirement.

24. The computer-implemented method of any of claims 1-21, wherein each of the plurality of binding sites satisfies a leading nucleotide constraint and a trailing nucleotide constraint.

25. The computer-implemented method of claim 24, wherein the identifying includes selecting the plurality of fragments using a pre-built nucleotide index for the given genome.

26. The computer-implemented method of any of claims 1-21, wherein the determining includes setting a specificity threshold and disregarding any binding the specificity of which does not exceed the specificity threshold.

27. The computer-implemented method of any of claims 1-21, wherein the scoring function generates a higher score when a smaller number of consecutive protein di-residues that bind to a “T” or an “A” nucleotide or to a “G” or “a “C” nucleotide, or a certain range for a length of the corresponding binding site.

28. The computer-implemented method of any of claims 1-21, wherein the scoring function associates a weight with at least one of the conditions (a) through (g) in computing a score.

29. The computer-implemented method of any of claims 1-21, wherein the output information includes one of the plurality of protein di-residue sequences, a number of binding sites for the protein di-residue sequence in the DNA region or the given genome, or a start position for each of the binding sites in the DNA region or the given genome.

30. The computer-implemented method of any of claims 1-21, further comprising:
identifying a second plurality of binding sites to the other side of the cleavage position within the DNA region;
determining a second plurality of protein di-residue sequences for a second plurality of the proteins to bind to the second plurality of binding sites based on the specificity information; and
assigning a score to each of the second plurality of protein di-residue sequences with the scoring function.

31. The computer-implemented method of claim 30, further comprising:
repeating the identifying, the determining, and the assigning for a complementary DNA sequence of the input DNA sequence,
wherein the output information includes one of the second plurality of protein di-residue sequences, a number of binding sites for the protein di-residue sequence in the DNA region or the given genome, or a start position for each of the binding sites in the DNA region or the given genome.

32. The computer-implemented method of claim 31, further comprising:
selecting a first protein di-residue sequence out of the plurality of protein di-residue sequences and a second protein di-residue sequence out of the second plurality of protein di-residue sequences based on the assigned scores,
wherein the first protein di-residue sequence has a binding site that is a certain distance away to the first side of the cleavage position and the second protein di-residue sequence has a binding site that is the certain distance away to the other side of the cleavage location; and
generating information regarding the selections of the first protein di-residue sequence and the second protein di-residue sequence.

33. The computer-implemented method of any of claims 1-21,
wherein each of the proteins is a transcription activator-like effector nuclease, and
wherein each of the protein di-residue sequences specifies the di-residues for the 12^{th} and the 13^{th} positions of the loops in the transcription activator-like effector nuclease.

34. The computer-implemented method of any of claims 1-21, further comprising receiving the input information from a client device over a network, and sending the output information to the client device over the network.

35. The computer-implemented method of claim 34, wherein the client device is a desktop computer, a laptop computer, a tablet, a cellular phone, or a wearable device.

36. A non-transitory computer-readable storage medium with instructions stored thereon that, when executed by a computing system, cause the computing system to perform a method of determining protein sequences for genome engineering, the method comprising:
receiving input information regarding an input DNA sequence for a DNA region in a given genome containing binding sites for proteins and a cleavage position for the proteins within the DNA region;
identifying a plurality of fragments of the input DNA sequence respectively corresponding to a plurality of the binding sites to a first side of the cleavage position;
determining a plurality of protein di-residue sequences for a plurality of the proteins to bind to the plurality of binding sites based on specificity information related to binding of protein di-residues to DNA bases;
assigning a score to each of the plurality of protein di-residue sequences with a scoring function that generates a score based on at least one of the following conditions of the protein di-residue sequence:
(a) TALE length or number of repeats;
(b) spacer length;
(c) last repeat variable dinucleotide (RVD);
(d) GC content of RVDs;
(e) first RVDs;
(f) uniqueness of binding sites in the given genome; or
(g) number of mononucleotide repeats; and
sending output information regarding the plurality of protein di-residue sequences, including the assigned scores.

37. The non-transitory computer-readable storage medium of claim 36, the method further comprising:
computing a number of binding sites within the given genome for each of the plurality of protein di-residue sequences,
wherein the plurality of conditions includes fewer binding sites within the given genome.

38. The non-transitory computer-readable storage medium of claim 37, wherein the computing is performed based on the specificity information.

39. The non-transitory computer-readable storage medium of claim 36, wherein the conditions include a binding site having more “G” or “C” nucleotides.

40. The non-transitory computer-readable storage medium of claim 36, wherein the conditions include a protein di-residue that binds with a higher specificity or a protein di-residue that binds with a higher efficiency in promoting protein activity.

41. A system for making nucleases for genome engineering, comprising:
an apparatus that develops proteins;
a memory; and
at least one processor in communication with the memory and the apparatus, the processor configured to perform:
receiving input information regarding an input DNA sequence for a DNA region in a given genome containing binding sites for proteins and a cleavage position for the proteins within the DNA region;
identifying a plurality of fragments of each of the input DNA sequence and a complementary DNA sequence of the input DNA sequence respectively corresponding to a plurality of the binding sites to each of the two sides of the cleavage position within the DNA region;
determining a plurality of protein di-residue sequences for a plurality of the proteins to bind to the plurality of binding sites based on specificity information related to binding of protein di-residues to DNA bases;
assigning a score to each of the plurality of protein di-residue sequences with a scoring function that generates a score based on at least one of the following conditions of the protein di-residue sequence:
(a) TALE length or number of repeats;
(b) spacer length;
(c) last repeat variable dinucleotide (RVD);
(d) GC content of RVDs;
(e) first RVDs;
(f) uniqueness of binding sites in the given genome; or
(g) number of mononucleotide repeats; and
selecting, based on the assigned scores, a first protein di-residue sequence out of the pluralities of protein di-residue sequences corresponding to a protein that bind to the input DNA sequence to a first side of the cleavage position and a second protein di-residue sequence out of the pluralities of protein di-residue sequences that bind to the complementary DNA sequence to the other side of the cleavage position; and
causing to display information regarding the first protein di-residue sequence and the second di-residue sequence,
wherein the apparatus develops proteins based on the first and the second di-residue sequences.

42. A computer-implemented method of determining protein sequences for genome engineering, comprising:
receiving input information regarding an input DNA sequence for a DNA region in a given genome containing binding sites for proteins and a cleavage position for the proteins within the DNA region;
identifying a plurality of fragments of the input DNA sequence respectively corresponding to a plurality of the binding sites to a first side of the cleavage position;
determining a plurality of protein di-residue sequences for a plurality of the proteins to bind to the plurality of binding sites based on specificity information related to binding of protein di-residues to DNA bases;
assigning a score to each of the plurality of protein di-residue sequences based on (1) a binding strength of initial protein di-residues, (2) a percentage of protein di-residues that bind to “G” or “C” nucleotides, or (3) a presence of consecutive protein di-residues that bind to “G” or “C” nucleotides or that bind to “A” or “T” nucleotides, in the protein di-residue sequence; and
generating output information regarding the plurality of protein di-residue sequences, including the assigned scores.

43. The method of claim 42, wherein the assigning includes calculating a score based on each of (1), (2), and (3), and determining a weighted average.

44. The method of claim 42, wherein a higher score is assigned when more of a predetermined number of the initial protein di-residues form a strong bond with a target nucleotide.

45. The method of claim 42, wherein a higher score is assigned when a larger percentage of the protein di-residues bind to “G” or “C” nucleotides.

46. The method of claim 42, wherein a higher score is assigned when no more than a first predetermined number of consecutive protein di-residues bind to “G” or “C” nucleotides and no more than a second predetermined number of consecutive protein di-residues bind to “A” or “T” nucleotides.

47. The method of claim 42, wherein a higher score is assigned when a length of the corresponding binding site falls in a first predetermined range or a length of a region between the corresponding binding site and the cleavage position falls in a second predetermined range.

48. The method of any of claims 42-47, further comprising receiving the input information from a client device over a network, and sending the output information to the client device over the network.

49. A high-throughput method of generating a nucleic acid construct containing a plurality of polynucleotides of interest, comprising:
a) assembling a first plurality of polynucleotides of interest in a first reaction mixture comprising a plurality of first destination vectors;
b) incorporating the first plurality of polynucleotides of interest into at least one first destination vector from the plurality of first destination vectors by a nucleic acid incorporation process to generate at least one first expression vector, wherein the at least one first expression vector comprises a first polynucleotide unit, and wherein the first polynucleotide unit comprises the first plurality of polynucleotides of interest;
c) incubating the first reaction mixture comprising the at least one first expression vector from step b) with a first restriction enzyme to remove a first destination vector that fails to incorporate the first plurality of polynucleotides of interest;
d) repeating steps a) to c) with a second plurality of polynucleotides of interest and a plurality of second destination vectors to generate at least one second expression vector, wherein the at least one second expression vector comprises a second polynucleotide unit, and wherein the second polynucleotide unit comprises the second plurality of polynucleotides of interest;
e) assembling the at least one first expression vector and the at least one second expression vector with a third destination vector in a second reaction mixture; and
f) incorporating the first polynucleotide unit and the second polynucleotide unit from the at least one first expression vector and the at least one second expression vector into the third destination vector by said nucleic acid incorporation process to generate the nucleic acid construct containing a plurality of polynucleotides of interest.

50. The method of claim 49, wherein the first restriction enzyme comprises BsaI or BsaI-HF.

51. The method of claim 49, further comprising incubating the first reaction mixture of step c) with a deoxyribonuclease.

52. The method of claim 49, wherein the incubating of step c) is for at least 30 minutes, at least 40 minutes, at least 50 minutes, at least 60 minutes, at least 70 minutes, at least 80 minutes, at least 90 minutes, at least 2 hours, at least 3 hours, at least 4 hours, at least 5 hours, at least 6 hours, at least 10 hours, at least 12 hours, or more.

53. The method of claim 49, wherein the incubating of step c) is at a temperature of 37° C.

54. The method of claim 49, wherein the incubating of step c) further comprises a transformation step, a culturing step, and a plasmid harvesting step.

55. The method of claim 54, wherein the plasmid obtained from the plasmid harvesting step is further quantified by a spectrophotometric method.

56. The method of claim 49, further comprising incubating the second reaction mixture after step f) with a second restriction enzyme to remove a third destination vector that fails to incorporate the first polynucleotide unit and the second polynucleotide unit.

57. The method of claim 56, wherein the second restriction enzyme comprises BsaI or BsaI-HF.

58. The method of claim 49 or 56, further comprising incubating the second reaction mixture after step f) with a deoxyribonuclease.

59. The method of claim 49 or 56, wherein the incubating of the second reaction mixture after step f) is for at least 30 minutes, at least 40 minutes, at least 50 minutes, at least 60 minutes, at least 70 minutes, at least 80 minutes, at least 90 minutes, at least 2 hours, at least 3 hours, at least 4 hours, at least 5 hours, at least 6 hours, at least 10 hours, at least 12 hours, or more.

60. The method of claim 49 or 56, wherein the incubating of the second reaction mixture after step f) is at a temperature of 37° C.

61. The method of claim 49 or 56, wherein the incubating further comprises a transformation step, a culturing step, and a plasmid harvesting step.

62. The method of claim 49, wherein the nucleic acid incorporation process comprises at least one round of a digestion step and a ligation step.

63. The method of claim 49, wherein the nucleic acid incorporation process comprises 2, 3, 4, 5, 6, 7, 8, 9, 10, 15, 20, or more rounds of a digestion step and a ligation step.

64. The method of claim 62 or 63, wherein the digestion step is at 37° C.

65. The method of claim 62 or 63, wherein the ligation step is at 16° C.

66. The method of any one of the claims 62-64, wherein the time for the digestion step is at least 2, 3, 4, 5, 6, 7, 8, 9, 10, 15, 30, or more minutes per round.

67. The method of any one of the claim 62, 63, or 65, wherein the time for the ligation step is 5, 6, 7, 8, 9, 10, 15, 30, 45, 60, or more minutes per round.

68. The method of any one of the claim 49 or 62-67, wherein the nucleic acid incorporation process further comprises a background reduction step.

69. The method of claim 68, wherein the background reduction step occurs after at least one round of a digestion step and a ligation step.

70. The method of claim 68 or 69, wherein the background reduction step occurs at a temperature of 45° C., 50° C., 55° C., 60° C., or higher.

71. The method of any one of the claims 68-70, wherein the time for the background reduction step is 5, 10, 15, 20, or more minutes.

72. The method of any one of the claim 49 or 62-71, wherein the nucleic acid incorporation process further comprises a heat inactivation step.

73. The method of claim 72, wherein the heat inactivation step occurs at a temperature of 65° C., 70° C., 75° C., 80° C., 85° C., 90° C., or higher.

74. The method of claim 72 or 73, wherein the time for the heat inactivation step is 5, 10, 15, 20, or more minutes.

75. The method of any one of the claims 49-74, wherein the first plurality of polynucleotides of interest comprises a plurality of TAL effector repeat modules or a plurality of zinc-binding repeat modules.

76. The method of claim 75, wherein the first plurality of polynucleotides of interest comprises a plurality of TAL effector repeat modules.

77. The method of any one of the claims 49-74, wherein the first plurality of polynucleotides of interest comprises a plurality of polynucleotides for generating a fusion polypeptide or a plurality of polynucleotides in which each polynucleotide encodes a portion of a protein of interest.

78. The method of claim 49, wherein the second plurality of polynucleotides of interest comprises a plurality of TAL effector repeat modules or a plurality of zinc-binding repeat modules.

79. The method of claim 78, wherein the second plurality of polynucleotides of interest comprises a plurality of TAL effector repeat modules.

80. The method of claim 49, wherein the second plurality of polynucleotides of interest comprises a plurality of polynucleotides for generating a fusion polypeptide or a plurality of polynucleotides in which each polynucleotide encodes a portion of a protein of interest.

81. The method of any one of the claim 49, 75, or 76, wherein the incorporating in step b) further comprises incubating the plurality of TAL effector repeat modules and the at least one first destination vector in the first reaction mixture for a first time period.

82. The method of any one of the claim 49, 75, or 76, wherein the incorporating in step b) further comprises culturing the plurality of TAL effector repeat modules and the at least one first destination vector for a second time period to generate a first TAL effector repeat containing vector.

83. The method of any one of the claim 49, 78, or 79, wherein step d) further comprises generating a second TAL effector repeat containing vector from a second plurality of TAL effector repeat modules and the at least one second destination vector.

84. The method of any one of the claim 49, 75, 76, 78, 79, or 81-83, wherein the incorporating in step f) further comprises incubating the first and the second TAL effector repeat containing vectors and the third destination vector in the second reaction mixture for a third time period.

85. The method of any one of the claim 49, 75, 76, 78, 79, or 81-84, wherein the incorporating in step f) further comprises culturing the first and the second TAL effector repeat containing vectors and the third destination vector for a fourth time period to generate a transcription activator-like (TAL) effector endonuclease monomer.

86. The method of any one of the claim 49, 75, 76, 78, 79, or 81-85, wherein the transcription activator-like (TAL) effector endonuclease monomer further comprises a FokI endonuclease domain and optionally a linker region.

87. The method of any one of the claim 49, 75, 76, 78, 79, or 81-86, wherein the transcription activator-like (TAL) effector endonuclease monomer further comprises a N-cap and a C-cap.

88. The method of any one of the claim 49, 75, 76, 78, 79, or 81-87, wherein the transcription activator-like (TAL) effector endonuclease monomer further comprises a C-terminal half-repeat.

89. The method of claim 88, wherein the C-terminal half-repeat comprises 15, 20, 21, 22, 23, 24, 25, 26, 27, 28, 29, 30, 35, or 40 amino acid residues.

90. The method of claim 88 or 89, wherein a sequence encoding the C-terminal half-repeat is present within the third destination vector.

91. The method of any one of the claim 49, 75, 76, 78, 79, or 81-89, wherein the transcription activator-like (TAL) effector endonuclease monomer further comprises a T base recognizing repeat variable-diresidue (RVD) at the N-terminal portion of the TAL effector repeat modules, at the C-terminal portion of the TAL effector repeat modules, or at both termini.

92. The method of any one of the claim 49, 75, 76, 78, 79, or 81-91, wherein the insertion of the TAL effector repeat modules removes a LacZ portion of the second vector.

93. The method of any one of the claim 49, 75, 76, 78, 79, or 81-92, wherein the plurality of TAL effector repeat modules comprises at least 1, 2, 3, 4, 5, 6, 7, 8, 9, 10, 11, 12, 13, 14, 15, 16, 17, 18, 19, 20, 21, 22, 23, 24, 25, 30, or more TAL effector repeat modules.

94. The method of any one of the claim 49, 75, 76, 78, 79, or 81-93, wherein each of the plurality of TAL effector repeat modules comprises a repeat variable-diresidue (RVD).

95. The method of claim 94, wherein the repeat variable-diresidue (RVD) comprises HD, NG, NI, NK, or NH.

96. The method of any one of the claims 49-95, wherein the first destination vector is pFUS vector.

97. The method of any one of the claims 49-95, wherein the first destination vector is pUC18 or pUC19 vector.

98. The method of any one of the claims 49-97, wherein the second destination vector is pFUS vector.

99. The method of any one of the claims 49-97, wherein the second destination vector is pUC18 or pUC19 vector.

100. The method of any one of the claims 49-99, wherein the third destination vector is pVax vector.

101. The method of any one of the claims 49-100, wherein the volume of the first reaction mixture is 2 μL.

102. The method of any one of the claims 49-100, wherein the volume of the second reaction mixture is 2 μL.

103. The method of claim 49, wherein the assembling of step a) and step e) are by an acoustic process.

104. The method of claim 103, wherein the acoustic process is generated by a Labcyte Echo 550 high-throughput acoustic liquid handler instrument.

105. A transcription activator-like (TAL) effector endonuclease monomer generated by the steps of:
a) assembling a first plurality of TAL effector repeat sequences in a first reaction mixture comprising a plurality of first destination vectors;
b) incorporating the first plurality of TAL effector repeat sequences into at least one first destination vector from the plurality of first destination vectors by a nucleic acid incorporation process to generate at least one first expression vector, wherein the at least one first expression vector comprises a first TAL effector repeat unit and wherein the first TAL effector repeat unit comprises the first plurality of TAL effector repeat sequences;
c) incubating the first reaction mixture comprising the at least one first expression vector from step b) with a first restriction enzyme to remove a first destination vector that fails to incorporate the first plurality of TAL effector repeat sequences;
d) repeating steps a) to c) with a second plurality of TAL effector repeat sequences and a plurality of second destination vectors to generate at least one second expression vector, wherein the at least one second expression vector comprises a second TAL effector repeat unit and wherein the second TAL effector repeat unit comprises the second plurality of TAL effector repeat sequences;
e) assembling the at least one first expression vector and the at least one second expression vector with a third destination vector in a second reaction mixture; and
f) incorporating the first TAL effector repeat unit and the second TAL effector repeat unit from the at least one first expression vector and the at least one second expression vector into the third destination vector by said nucleic acid incorporation process to generate the nucleic acid construct containing the transcription activator-like (TAL) effector endonuclease monomer.

106. A high-throughput method of generating a nucleic acid construct containing a plurality of polynucleotides of interest, comprising:
a) assembling a first plurality of polynucleotides of interest and a plurality of first destination vectors in a first reaction mixture by an acoustic process;
b) incorporating the first plurality of polynucleotides of interest into at least one first destination vector from the plurality of first destination vectors by a nucleic acid incorporation process to generate at least one first expression vector, wherein the at least one first expression vector comprises a first polynucleotide unit and wherein the first polynucleotide unit comprises the first plurality of polynucleotides of interest;
c) repeating steps a) and b) with a second plurality of polynucleotides of interest and a plurality of second destination vectors to generate at least one second expression vector, wherein the at least one second expression vector comprises a second polynucleotide unit and wherein the second polynucleotide unit comprises the second plurality of polynucleotides of interest;
d) assembling the at least one first expression vector and the at least one second expression vector with a third destination vector in a second reaction mixture by said acoustic process; and
e) incorporating the first polynucleotide unit and the second polynucleotide unit from the at least one first expression vector and the at least one second expression vector into the third destination vector by said nucleic acid incorporation process to generate the nucleic acid construct containing a plurality of polynucleotides of interest.

107. The method of claim 106, further comprising a treating step after step b) but prior to step d), wherein the treating step comprises incubating the first reaction mixture from step b) with a first restriction enzyme to remove a first destination vector that fails to incorporate the first plurality of polynucleotides of interest.

108. The method of claim 107, wherein the first restriction enzyme comprises BsaI or BsaI-HF.

109. The method of claim 107, wherein the treating step further comprises incubating the first reaction mixture with a deoxyribonuclease.

110. The method of claim 109, wherein the incubating is for at least 30 minutes, at least 40 minutes, at least 50 minutes, at least 60 minutes, at least 70 minutes, at least 80 minutes, at least 90 minutes, at least 2 hours, at least 3 hours, at least 4 hours, at least 5 hours, at least 6 hours, at least 10 hours, at least 12 hours, or more.

111. The method of claim 109, wherein the incubating is at a temperature of 37° C.

112. The method of claim 107, wherein the treating step further comprises a transformation step, a culturing step, and a plasmid harvesting step.

113. The method of claim 112, wherein the plasmid obtained from the plasmid harvesting step is further quantified by a spectrophotometric method.

114. The method of claim 106, further comprising a treating step after step e), wherein the treating step comprises incubating the second reaction mixture from step e) with a second restriction enzyme to remove a third destination vector that fails to incorporate the first polynucleotide unit and the second polynucleotide unit.

115. The method of claim 114, wherein the second restriction enzyme comprises BsaI or BsaI-HF.

116. The method of claim 114, wherein the treating step further comprises incubating the second reaction mixture after step f) with a deoxyribonuclease.

117. The method of claim 114, wherein the incubating is for at least 30 minutes, at least 40 minutes, at least 50 minutes, at least 60 minutes, at least 70 minutes, at least 80 minutes, at least 90 minutes, at least 2 hours, at least 3 hours, at least 4 hours, at least 5 hours, at least 6 hours, at least 10 hours, at least 12 hours, or more.

118. The method of claim 114, wherein the incubating is at a temperature of 37° C.

119. The method of claim 114, wherein the treating step further comprises a transformation step, a culturing step, and a plasmid harvesting step.

120. The method of any one of the claims 106-119, wherein the first plurality of polynucleotides of interest comprises a plurality of TAL effector repeat modules or a plurality of zinc-binding repeat modules.

121. The method of claim 120, wherein the first plurality of polynucleotides of interest comprises a plurality of TAL effector repeat modules.

122. The method of any one of the claims 106-119, wherein the first plurality of polynucleotides of interest comprises a plurality of polynucleotides for generating a fusion polypeptide or a plurality of polynucleotides in which each polynucleotide encodes a portion of a protein of interest.

123. The method of claim 106, wherein the second plurality of polynucleotides of interest comprises a plurality of TAL effector repeat modules or a plurality of zinc-binding repeat modules.

124. The method of claim 123, wherein the second plurality of polynucleotides of interest comprises a plurality of TAL effector repeat modules.

125. The method of claim 106, wherein the second plurality of polynucleotides of interest comprises a plurality of polynucleotides for generating a fusion polypeptide or a plurality of polynucleotides in which each polynucleotide encodes a portion of a protein of interest.

126. The method of any one of the claim 106, 120, or 121, wherein the incorporating in step b) further comprises incubating the plurality of TAL effector repeat modules and the at least one first destination vector in the first reaction mixture for a first time period.

127. The method of any one of the claim 106, 120, or 121, wherein the incorporating in step b) further comprises culturing the plurality of TAL effector repeat modules and the at least one first destination vector for a second time period to generate a first TAL effector repeat containing vector.

128. The method of any one of the claim 106, 120, 121, 126, or 127, wherein step c) further comprises generating a second TAL effector repeat containing vector from a second plurality of TAL effector repeat modules and the at least one second destination vector.

129. The method of any one of the claim 106, 120, 121, or 126-128, wherein the incorporating in step e) further comprises incubating the first and the second TAL effector repeat containing vectors and the third destination vector in the second reaction mixture for a third time period.

130. The method of any one of the claim 106, 120, 121, or 126-128, wherein the incorporating in step e) further comprises culturing the first and the second TAL effector repeat containing vectors and the third destination vector for a fourth time period to generate a transcription activator-like (TAL) effector endonuclease monomer.

131. The method of any one of the claim 106, 120, 121, or 126-130, wherein the transcription activator-like (TAL) effector endonuclease monomer further comprises a FokI endonuclease domain and optionally a linker region.

132. The method of any one of the claim 106, 120, 121, or 126-131, wherein the transcription activator-like (TAL) effector endonuclease monomer further comprises a N-cap and a C-cap.

133. The method of any one of the claim 106, 120, 121, or 126-132, wherein the transcription activator-like (TAL) effector endonuclease monomer further comprises a C-terminal half-repeat.

134. The method of claim 133, wherein the C-terminal half-repeat comprises 15, 20, 21, 22, 23, 24, 25, 26, 27, 28, 29, 30, 35, or 40 amino acid residues.

135. The method of claim 133 or 134, wherein a sequence encoding the C-terminal half-repeat is present within the third destination vector.

136. The method of any one of the claim 106, 120, 121, or 126-135, wherein the transcription activator-like (TAL) effector endonuclease monomer further comprises a T base recognizing-repeat variable-diresidue (RVD) at the N-terminal portion of the TAL effector repeat modules, at the C-terminal portion of the TAL effector repeat modules, or at both termini.

137. The method of any one of the claim 106, 120, 121, or 126-136, wherein the insertion of the TAL effector repeat modules removes a LacZ portion of the second vector.

138. The method of any one of the claim 106, 120, 121, or 126-137, wherein the plurality of TAL effector repeat modules comprises at least 1, 2, 3, 4, 5, 6, 7, 8, 9, 10, 11, 12, 13, 14, 15, 16, 17, 18, 19, 20, 21, 22, 23, 24, 25, 30, or more TAL effector repeat modules.

139. The method of any one of the claim 106, 120, 121, or 126-138, wherein each of the plurality of TAL effector repeat modules comprises a repeat variable-diresidue (RVD).

140. The method of claim 139, wherein the repeat variable-diresidue (RVD) comprises HD, NG, NI, NK, or NH.

141. The method of claim 106, wherein the nucleic acid incorporation process comprises at least one round of a digestion step and a ligation step.

142. The method of claim 106, wherein the nucleic acid incorporation process comprises 2, 3, 4, 5, 6, 7, 8, 9, 10, 15, 20, or more rounds of a digestion step and a ligation step.

143. The method of claim 141 or 142, wherein the digestion step is at 37° C.

144. The method of claim 141 or 142, wherein the ligation step is at 16° C.

145. The method of any one of the claims 141-143, wherein the time for the digestion step is at least 2, 3, 4, 5, 6, 7, 8, 9, 10, 15, 30, or more minutes per round.

146. The method of any one of the claim 141, 142, or 144, wherein the time for the ligation step is 5, 6, 7, 8, 9, 10, 15, 30, 45, 60, or more minutes per round.

147. The method of any one of the claim 106 or 141-146, wherein the nucleic acid incorporation process further comprises a background reduction step.

148. The method of claim 147, wherein the background reduction step occurs after at least one round of a digestion step and a ligation step.

149. The method of claim 147 or 148, wherein the background reduction step occurs at a temperature of 45° C., 50° C., 55° C., 60° C., or higher.

150. The method of any one of the claims 147-149, wherein the time for the background reduction step is 5, 10, 15, 20, or more minutes.

151. The method of any one of the claim 106 or 147-150, wherein the nucleic acid incorporation process further comprises a heat inactivation step.

152. The method of claim 151, wherein the heat inactivation step occurs at a temperature of 65° C., 70° C., 75° C., 80° C., 85° C., 90° C., or higher.

153. The method of claim 151 or 152, wherein the time for the heat inactivation step is 5, 10, 15, 20, or more minutes.

154. The method of any one of the claims 106-153, wherein the first destination vector is pFUS vector.

155. The method of any one of the claims 106-153, wherein the first destination vector is pUC18 or pUC19 vector.

156. The method of any one of the claims 106-155, wherein the second destination vector is pFUS vector.

157. The method of any one of the claims 106-155, wherein the second destination vector is pUC18 or pUC19 vector.

158. The method of any one of the claims 106-157, wherein the third destination vector is pVax vector.

159. The method of any one of the claims 106-158, wherein the volume of the first reaction mixture is 2 μL.

160. The method of any one of the claims 106-159, wherein the volume of the second reaction mixture is 2 μL.

161. The method of claim 106, wherein the acoustic process is generated by a Labcyte Echo 550 high-throughput acoustic liquid handler instrument.

162. A transcription activator-like (TAL) effector endonuclease monomer generated by the steps of:
a) assembling a first plurality of TAL effector repeat sequences and a plurality of first destination vectors in a first reaction mixture by an acoustic process;
b) incorporating the first plurality of TAL effector repeat sequences into at least one first destination vector from the plurality of first destination vectors by a nucleic acid incorporation process to generate at least one first expression vector, wherein the at least one first expression vector comprises a first TAL effector repeat unit and wherein the first TAL effector repeat unit comprises the first plurality of TAL effector repeat sequences;
c) repeating steps a) and b) with a second plurality of TAL effector repeat sequences and a plurality of second destination vectors to generate at least one second expression vector, wherein the at least one second expression vector comprises a second TAL effector repeat unit and wherein the second TAL effector repeat unit comprises the second plurality of TAL effector repeat sequences;
d) assembling the at least one first expression vector and the at least one second expression vector with a third destination vector in a second reaction mixture by said acoustic process; and
e) incorporating the first TAL effector repeat unit and the second TAL effector repeat unit from the at least one first expression vector and the at least one second expression vector into the third destination vector by said nucleic acid incorporation process to generate the transcription activator-like (TAL) effector endonuclease monomer.

163. A method for making transcription activator-like effector nucleases (TALENs) for genome engineering, comprising:
determining, by a computer-implemented method according to any of claims 1-35, scores for a plurality of protein di-residue sequences corresponding to an input DNA sequence for a DNA region in a given genome containing binding sites for proteins and a cleavage position for the proteins within the DNA region;
selecting, based on the scores, a first protein di-residue sequence out of the plurality of protein di-residue sequences corresponding to a protein that bind to the input DNA sequence to a first side of the cleavage position and a second protein di-residue sequence out of the plurality of protein di-residue sequences that bind to the complementary DNA sequence to the other side of the cleavage position; and
producing the TALENs based on the first and the second di-residue sequences.
